SEQUENCE LISTING (7 sequences)

SEQ ID NO: 1 - DNA, 1482 bp, synthetic construct
  source 1..1482: mol_type = other DNA; note = monospecific anti-PSMA CAR
atggccctgcctgtgacagccctgctgctgcctctggctctgctgctgcatgccgctagacccggatccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagccccgaggtcgaagatggattattggggccagggaaccctggtcacagtctcgagcggtggaggcggttcaggcggaggtggcagcggcggtggcgggtcaacgcagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatatcgtgggcgagcaggtcgtgttcggcggagggaccaagctgaccgtcctatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcggcgcgcctaa

SEQ ID NO: 2 - protein, 493 aa, synthetic construct
MALPVTALLLPLALLLHAARPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAPRSKMDYWGQGTLVTVSSGGGGSGGGGSGGGGSTQSVLTQPPSASGTPGQRVTISCSGSSSNIGSNTVNWYQQLPGTAPKLLIYSNNQRPSGVPDRFSGSKSGTSASLAISGLQSEDEADYYCAAWDIVGEQVVFGGGTKLTVLSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRA

SEQ ID NO: 3 - DNA, 1494 bp, synthetic construct
  source 1..1494: mol_type = other DNA; note = monospecific anti-Nectin-2 CAR
atgagcctgcccgtgaccgccctgctgctgcccctggccctgctgctgcacgccgccagaccaggatcccaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagatatgggtggaaggatgctatggattattggggccagggaaccctggtcacagtctcgagcggtggaggcggttcaggcggaggtggcagcggcggtggcgggtcaacgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatcggcccaagcagaaggtggtgttcggcggagggaccaagctgaccgtcctatccggagccaccacaaccaagcccgtgctgcggaccccaagccctgtgcaccctaccggcaccagccagcctcagagacccgaggactgccggcctcggggcagcgtgaagggcaccggcctggacttcgcctgcgacttttgggtgctggtggtggttggtggagtcctggcttgctatagcttgctagtaacagtggcctttattattttctgggtgcctgcaggaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 4 - protein, 497 aa, synthetic construct
MSLPVTALLLPLALLLHAARPGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYGWKDAMDYWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDRPKQKVVFGGGTKLTVLSGATTTKPVLRTPSPVHPTGTSQPQRPEDCRPRGSVKGTGLDFACDFWVLVVVGGVLACYSLLVTVAFIIFWVPAGKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 2559 bp, synthetic construct
  source 1..2559: mol_type = other DNA; note = dual PSMA Nectin-2 CAR
atggccctgcctgtgacagccctgctgctgcctctggctctgctgctgcatgccgctagacccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagccccgaggtcgaagatggattattggggccagggaaccctggtcacagtctcgagcggtggaggcggttcaggcggaggtggcagcggcggtggcgggtcaacgcagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatatcgtgggcgagcaggtcgtgttcggcggagggaccaagctgaccgtcctatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcggcgcgccgaaggaagaggtagcctgctgacttgcggggacgtggaagagaacccagggccaatgagcctgcccgtgaccgccctgctgctgcccctggccctgctgctgcacgccgccagaccaggatcccaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagatatgggtggaaggatgctatggattattggggccagggaaccctggtcacagtctcgagcggtggaggcggttcaggcggaggtggcagcggcggtggcgggtcaacgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatcggcccaagcagaaggtggtgttcggcggagggaccaagctgaccgtcctatccggagccaccacaaccaagcccgtgctgcggaccccaagccctgtgcaccctaccggcaccagccagcctcagagacccgaggactgccggcctcggggcagcgtgaagggcaccggcctggacttcgcctgcgacttttgggtgctggtggtggttggtggagtcctggcttgctatagcttgctagtaacagtggcctttattattttctgggtgcctgcaggaaaacggggcagaaagaaactcctgtatatattcaaacaaccatttatgagaccagtacaaactactcaagaggaagatggctgtagctgccgatttccagaagaagaagaaggaggatgtgaactgtaa

SEQ ID NO: 6 - protein, 854 aa, synthetic construct
MALPVTALLLPLALLLHAARPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAPRSKMDYWGQGTLVTVSSGGGGSGGGGSGGGGSTQSVLTQPPSASGTPGQRVTISCSGSSSNIGSNTVNWYQQLPGTAPKLLIYSNNQRPSGVPDRFSGSKSGTSASLAISGLQSEDEADYYCAAWDIVGEQVVFGGGTKLTVLSGTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMSLPVTALLLPLALLLHAARPGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYGWKDAMDYWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDRPKQKVVFGGGTKLTVLSGATTTKPVLRTPSPVHPTGTSQPQRPEDCRPRGSVKGTGLDFACDFWVLVVVGGVLACYSLLVTVAFIIFWVPAGKRGRKKLLYIFKQPFMRPVQTTQEEDGCSCRFPEEEEGGCEL

SEQ ID NO: 7 - protein, 20 aa, synthetic construct
  source 1..20: note = Nectin 2 cancer-exposed epitope
RFTLVPSGRADGVTVTCKVE

(19) United States
(12) Patent Application Publication
(10) Pub. No.: US 2026/0356973 A1
(43) Pub. Date: Nov. 12, 2026

(54) DUAL TARGETING OF PSMA AND NECTIN2 IN PROSTATE CANCER

(71) Applicants: UNIVERSITE DE GENEVE, GENEVE (CH); FONDAZIONE PER L’ISTITUTO ONCOLOGICO DI RICERCA, Bellinzona (CH); HOPITAUX UNIVERSITAIRES DE GENEVE, GENEVE (CH)

(72) Inventors: Andrea ALIMONTI, Bosco Luganese (CH); Denis MIGLIORINI, Geneve (CH)

(21) Appl. No.: 19/486,771

(22) PCT Filed: May 23, 2024
(86) PCT No.: PCT/EP2024/064288

§ 371 Date: Nov. 21, 2025

(30) Foreign Application Priority Data
May 23, 2023 (EP) 23174810.4

(51) Int. Cl. C07K 16/30; A61K 40/11; A61K 40/31; A61K 40/42; A61P 35/00; C07K 14/705; C07K 16/28; C12N 5/0783

(52) U.S. Cl. CPC C07K 16/3069; A61K 40/11; A61K 40/31; A61K 40/421; A61K 40/4276; A61P 35/00; C07K 14/705; C07K 16/2803; C12N 5/0636; C07K 2319/02; C07K 2319/03; C12N 2510/00

(57) ABSTRACT

The present invention concerns dual-targeting of PSMA and a novel senescence marker, Nectin2, on prostate cancer cells by immunoresponsive cell therapy that uses dual-targeting CAR immunoresponsive cells, or dual-targeting CAR/synthetic Notch (synNotch) receptor immunoresponsive cells recognizing PSMA and Nectin2. The invention further concerns related nucleic acids, vectors, CARs, CAR/synNotch receptors, and immunoresponsive cells.

The present invention concerns dual-targeting of PSMA and a novel senescence marker, Nectin2, on prostate cancer cells by immunoresponsive cell therapy that uses dual-targeting CAR immunoresponsive cells, or dual-targeting CAR/synthetic Notch (synNotch) receptor immunoresponsive cells recognizing PSMA and Nectin2. The invention further concerns related nucleic acids, vectors, CARs, CAR/synNotch receptors, and immunoresponsive cells.

### BACKGROUND

Prostate cancer (PCa) is still a significant cause of cancer-related mortality and morbidity in men, being the second most diagnosed cancer and sixth leading cause of cancer death worldwide. Predisposition to prostate cancer development is high, with more than 70% of all cases being diagnosed in men over 65 years old. The development of prostate cancer is a multi-step process, starting with prostate hyperplasia and progressing to high-grade prostatic intraepithelial neoplasia (HGPIN) and eventually becoming invasive and metastatic.

Treatment is usually based on chemotherapy and radiotherapy with or without a castration-based strategy, but most treatments show limited duration of clinical and survival benefit due to primary and acquired resistance caused by tumor adaptability (Sumanasuriya, S. & Bono, J. D. Cold Spring Harb. Perspect. Med. 8, a030635 (2018)). Docetaxel and androgen deprivation therapy (ADT) are widely used for the treatment of prostate cancer. However, nearly all patients under ADT usually progress to castration-resistance prostate cancer (CRPC), which can eventually evolve into metastatic castration-resistant prostate cancer (mCRPC) (Nakazawa, M., Paller, C. & Kyprianou, N. Curr. Oncol. Rep. 19, 13 (2017); Saad, F. & Fizazi, K. Urology 86, 852-861 (2015)).

Therefore, current research aims at finding new therapeutic options and overcoming resistance mechanisms.

Besides, the induction of senescence, whether caused by traditional therapeutic methods or genetic mutation, plays a controversial role in cancer by both benefiting and harming tumor growth (Collado, M. Future Oncol. 6, 687-689 (2010)).

The therapeutic benefits of senescence induction come from the blockade of cellular proliferation and the activation of anti-tumoral immune responses, while the accumulation of senescent cells can stimulate tumor growth and angiogenesis, leading to relapse and therapy resistance (Hernandez-Segura, A. et al. Trends Cell Biol. 28, 436-453 (2018)). Eliminating senescent cells has become an interesting strategy for improving therapeutic efficacy. Senescent immune cells, particularly T cells, may also accumulate in the tumor microenvironment, a phenomenon known as immunosenescence, which can contribute to cancer development (Ye, J. et al. EMBO Mol. Med. 6, 1294-1311 (2014); Ye, J. et al. Blood 120, 2021-2031 (2012)). When cancer cells turn senescent, the amount of released SASP (senescence-associated secretory phenotype) components affects the recruitment of immune cells, and the effect of SASP on immune cells populations is considered a double-edged sword effect. The recruitment of T cells, macrophages, and NK cells by SASP can prevent tumor initiation and progression, but SASP can also increase immunosuppressive myeloid cells tumor infiltration. In the absence of tumor-derived factors, myeloid cells differentiate into dendritic cells, macrophages, or neutrophils, which can contribute to immune surveillance. However, in the presence of tumor-derived factors, these myeloid cells lose their capacity to differentiate and inhibit the function of other immune cells, creating an immunotolerant environment that allows tumor progression (Ohtani, N. Inflamm. Regen. 42, 11 (2022); Eggert, T. et al. Cancer Cell 30, 533-547 (2016)). Hence, the idea of a “one-two punch” approach has been proposed, in which a stable arrest of cellular proliferation in cancer cells is established using “pro-senescence” drugs, and then subsequent treatment with an agent able to kill senescent cells (senolytic drug) is administered (Wang, C. et al. Nature 574, 268-272 (2019); Sieben C. J. et al., Trends in Cell Biology, 28, Issue 9, 723-737 (2018)). Cellular senescence represents a promising target for improving the outcome of current therapies, and several senolytic agents have been identified and obtained from natural products to be used in combination with pro-senescence drugs.

Eliminating senescent cells has therefore emerged as a promising therapeutic strategy to prevent tumor relapse and metastases in different cancers. However, senescent tumor cells are difficult to kill since they upregulate several pro-survival pathways, and the current armamentarium of effective senolytics includes only a small number of compounds that are suitable for clinical trials (Zhu, Y. et al. Aging 9, 955-963 (2017); Zhu, Y. et al. Aging Cell 15, 428-435 (2016); Yousefzadeh M J et al. EBioMedicine, 36,18-28 (2018)).

The use of immune-checkpoint inhibitors (ICIs) has transformed cancer treatment by reactivating the immune system against malignant cells. However, only a few patients experience a durable response to ICIs, and resistance to therapy is widespread. Resistance to ICIs can be influenced by both tumor cell-extrinsic and intrinsic factors. Prostate cancer is known to have a weak immune response, with a low neoantigen load and a highly immunosuppressive microenvironment. A higher number of tumor-infiltrated lymphocytes (TILs) is a positive prognostic indicator in many types of cancer, including melanoma and breast cancer, but in prostate cancer, most of T-cells inside the tumor are CD4+ regulatory T cells (Tregs). Currently, ongoing clinical trials are evaluating the use of ICIs alone or in combination therapy for metastatic hormone-sensitive and castration-resistant prostate cancer. Synthetic immunology, specifically CAR-T cell therapy, is another strategy being explored for prostate cancer treatment. Immune checkpoints inhibitors (ICIs) have revolutionized the current approaches to treating cancer. In healthy condition, immune checkpoints prevent the activation of a strong immune response against normal cells in the body. This regulation is based on the specific binding of immune checkpoints expressed by immune cells to their partner binding protein. In cancer condition, the block of this interaction through ICIs results in the reactivation of the immune system against malignant cells.

Nowadays, many antibodies and small molecules targeting immune checkpoints such as CTLA-4, PD-1, PD-L1, TIGIT, TIM3 and CD47 are in clinical development. By far, the most widely used ICIs are antibodies targeting CTLA-4 (e.g. Ipilimumab), PD1 (Pembrolizumab, Nivolumab) and PD-L1 (e.g. Aterolizumab). Although the application of ICIs has improved patient outcomes across different tumor types, only a few percentages of patients achieve a durable response. Indeed, the onset of resistance mechanism to immune checkpoints inhibitors is a widespread clinical event. Even among those patients with melanoma, which present one of the highest positive response to ICIs, more than the 60% of them do not show an objective response to anti-PD-1 therapy (Ott, P. A. et al. J. Clin. Oncol. 37, 318-327 (2019)).

The cause of ICIs resistance development relies on both tumor cell-extrinsic and intrinsic factors. Among the tumor extrinsic factors, the microbiome, the PD-L1 expression levels on immune cells and the tumoral and peripheral immune cell composition play an important role in deciding the therapeutic outcome. On the other hand, epigenetic variations, mutational burden and neoantigen expression are all tumor cell-intrinsic feature which can direct the patient whether the response to ICIs therapy will be effective or not (Bagchi, S., Yuan, R. & Engleman, E. G. Annu. Rev. Pathol. 16, 223-249 (2021)).

Prostate cancer is considered as an immunologically cold disease, and a weak immune response towards it characterizes it. Indeed, it usually presents T-cell exhaustion, a low neoantigen load and a highly immunosuppressive microenvironment (de Bono, J. S. et al. Nat. Rev. Cancer 20, 455-469 (2020); Krueger, T. E., Thorek, D. L. J., Meeker, A. K., Isaacs, J. T. & Brennen, W. N. The Prostate 79, 320-330 (2019)). The tumor microenvironment is mainly constituted by Tregs, M2-polarized tumor-associated macrophages (TAMs) and myeloid-derived suppressor cells (MDSCs), and all of them generally present an immunosuppressive phenotype (Krueger, T. E., Thorek, D. L. J., Meeker, A. K., Isaacs, J. T. & Brennen, W. N. The Prostate 79, 320-330 (2019)). Specifically, MDSCs have been found to have a potent immunosuppressive role in castration-resistant prostate cancer (CRPC), where the interleukin-23 (IL-23) produced by MDSCs can regulate castration resistance by triggering androgen receptor signalling (Calcinotto, A. et al. Nature 559, 363-369 (2018)). Within this frame of reference, some pre-clinical studies already showed the beneficial effect of anti-MDSCs and ICIs combination therapy to increase the immune checkpoint blockade efficacy in CRPC models (Calcinotto, A. et al. Nature 559, 363-369 (2018); Lu, X. et al. Nature 543, 728-732 (2017)).

As previously mentioned, a higher number of tumour-infiltrated lymphocytes (TILs) is a prognostic indicator of a better outcome in many types of cancer, such as melanoma and breast cancer. In PCa, besides the low percentage of TILs, most of the T-cells inside the tumor in mCRPC resulted in being CD4+ regulatory T cells (Tregs), and the low abundant CD8+ T cells usually present an exhausted phenotype together with the expression of inhibitory receptors such as PD-1, LAG-3 and TIM-3 (Nava Rodrigues, D. et al. J. Clin. Invest. 128, 4441-4453 (2018); Brady, L. et al. Nat. Commun. 12, 1426 (2021); He, M. X. et al. Nat. Med. 27, 426-433 (2021)).

Currently, there are many ongoing clinical trials on ICIs in locally advanced or metastatic hormone-sensitive and castration-resistant prostate cancer, where ICI are used alone or in combination therapy (Rebuzzi, S. E. et al. Cancers 14, 1245 (2022)). Overall, no consistent results in terms of overall survival have been found. Patients who partially benefit from anti-PD1 therapy, such as nivolumab and pembrolizumab, were found to be characterized by a CDK12-altered tumor. Indeed, CDK12-mutated PCas are typically linked with a poor prognosis but present increased neoantigen load and lymphocytic infiltration. Besides, many studies assessing a combination therapy of ICI with standard therapies underline a better response in those patients with specific pathway aberrations (e.g. AR-V7 variant, HRD), CDK12-inactivated tumors and MSI-high tumors (Rebuzzi, S. E. et al. Cancers 14, 1245 (2022)). These clinical trials underline the need to identify novel therapeutic targets and strategies to boost the immune response in PCa patients based on the tumour's genetic signature.

Synthetic immunology represents an alternative strategy to ICIs. The primary goal of synthetic immunology is to genetically engineer immune cells to be used as therapeutic agents for diseases difficult to treat, such as cancer and autoimmunity. Two main strategies have been adopted for synthetically boosting T cell immunity, chimeric antigen receptors (CAR) engineered T cells and T cells engaging bispecific (or tri specific) antibodies. Both approaches encourage T cell activation by promoting the recognition of tumor-associated antigens (TAA) expressed on the tumor cell surface and triggering intracellular activation of T cell receptor (TCR). Most importantly, since synthetic T cells recognize extracellular TAA, these strategies do not rely on antigen processing and presentation by MHC complexes, thus bypassing all the immune evasion mechanisms deployed by the tumor (Sterner, R. C. & Sterner, R. M. Blood Cancer J. 11, 1-11 (2021); Johnson, L. A. & June, C. H. Cell Res. 27, 38-58 (2017); Dimitrios L. Wagner, et al. Clinical oncology, Vol. 18 (6), p.379-393. 2021)). Today, CAR-T cells and T cell-engaging bispecific antibodies have shown potent effects in patients with hematologic cancers, although limited effect against solid cancers (Dimitrios L. Wagner, et al. Clinical oncology, Vol. 18 (6), p.379-393. 2021)). The major obstacle of using T cell-engaging antibodies in solid tumors is the poor availability of tumour-specific targets. Most solid tumor TAAs are also expressed on non-malignant primary cells in critical tissues, even at a lower level.

For solid tumors, including prostate cancer (PCa), in addition, CAR T cells face a range of highly immunosuppressive barriers in the solid tumor microenvironment (TME). For example, inhibitory molecules such as TGFb and chronic exposure to antigens, which can render them exhausted and limit their persistence and effector function.

Fewer than 10% of patients refractory to chemotherapy benefit from immune checkpoint blockade immunotherapy (Markowski, M. C. et al. The Prostate 80, 407-411 (2020)). Hence, mCRPC represents a critical unmet medical need.

Second-generation PSMA-targeting CAR T cells expressing a dominant negative TFGFb receptor can have impressive and durable antitumor activity, including complete responses in some patients with mCRPC (Narayan, V. et al. Nat. Med. 28, 724-734 (2022)). However, the utility of this therapeutic strategy may be limited by multiple factors. First, a high disease burden account for significant cytokine release syndrome (CRS), bring about mortality with these potent CAR T cells. This is, in spite of the state-of-the-art patient management and thus remains an important clinical challenge (Cosenza, M., et al. Int. J. Mol. Sci. 22, 7652 (2021)). In addition, heterogeneous expression and/or downregulation of PSMA can cause tumor escape (Anderson, J., et al. Clin. Cancer Res. 28, 3196-3206 (2022)). Moreover, the suppressive microenvironment of mCRPC (Drake, C., Sharma, P. & Gerritsen, W. Oncogene 33, 5053-5064 (2014); Laccetti, A. L. & Subudhi, S. K. Curr. Opin. Urol. 27, 566-571 (2017); Vigano, S. et al. Front. Immunol. 10, (2019)) restricts the CAR T cell function (Paschalis, A. et al. Eur. Urol. 76, 469-478 (2019)).

Understanding the adaptive immune response in advanced prostate cancer (APC) has become crucial, especially after the anti-CTLA4 (ipilimumab) therapy failed in phase III trials (Beer, T. M. et al. J. Clin. Oncol. Off. J. Am. Soc. Clin. Oncol. 35, 40-47 (2017)). Also, PD-L1, the main target of existing immunotherapies, is infrequently expressed on prostate cancer (Haffner, M. C. et al. Am. J. Pathol. 188, 1478-1485 (2018)). Lately, the largest clinical trial testing anti-CTLA-4 plus anti-PD-1 in CRPC did not significantly ameliorate the patient's clinical outcome (Sharma, P. et al. Cancer Cell 38, 489-499.e3 (2020)).

### SUMMARY OF THE INVENTION

The inventors hypothesized that further development of effective CAR therapy for CRPC should include the identification of robust tumor target antigens that are stably and homogeneously expressed, incorporating safety mechanisms to mitigate toxicity, as well as strategies to overcome detrimental barriers to CAR T cells in the tumor microenvironment (TME).

The inventors therefore aimed to identify novel immune checkpoints in prostate cancer and exploit them as a new target for developing immunotherapeutic strategies for prostate cancer. Through analysis of bulk-RNA and single-cell data from human patients, the transmembrane protein Nectin2 was identified as upregulated in epithelial prostate cancer cells. Nectin2 has immunomodulatory functions and interacts with receptors expressed by T and NK cells. The upregulation of Nectin2 was found to be even higher upon senescence induction in vitro and in vivo models of therapy-induced senescence and PTEN-loss-induced cellular senescence.

Based on these findings, Nectin2 represents an exploitable target to remove senescent cancer cells from the tumor and reactivate the immune response. Senolytic Dual CAR-T cells targeting Nectin2 and the prostate-specific membrane antigen (PSMA) were generated and shown to be specific and selective in multiple in vitro assays.

The invention relates to a nucleic acid that encodes:
a. a first chimeric antigen receptor (CAR) that comprises (i) an antigen binding domain that binds prostate-specific membrane antigen (PSMA), (ii) a transmembrane domain, and (iii) an intracellular signaling domain comprising an activation domain, and a second CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain, and a second CAR that comprises (i) an antigen binding domain that binds PSMA, (ii) a transmembrane domain, and (iii) an intracellular signaling domain comprising a co-stimulatory domain.

The invention also relates to a pair of nucleic acids, comprising:
a. a first nucleic acid that encodes a CAR that comprises (i) an antigen binding domain that binds prostate-specific membrane antigen (PSMA), (ii) a transmembrane domain, and (iii) an intracellular signaling domain comprising an activation domain, and a second nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising an activation domain, and a second nucleic acid that encodes a CAR that comprises (i) an antigen binding domain that binds PSMA, (ii) a transmembrane domain, and (iii) an intracellular signaling domain comprising a co-stimulatory domain.

The invention further relates to a pair of nucleic acids, comprising:
a. a first nucleic acid that encodes a synthetic Notch (synNotch) receptor comprising an antigen binding domain that binds prostate-specific membrane antigen (PSMA); and a second nucleic acid that encodes a chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain and optionally a co-stimulatory domain; or
b. a first nucleic acid that encodes a synthetic Notch (synNotch) receptor comprising an antigen binding domain that binds an antigen, other than PSMA, which is a marker of senescence in prostate cancer cells; and a second nucleic acid that encodes a chimeric antigen receptor (CAR) that comprises (i) an antigen binding domain that binds PSMA, (ii) a transmembrane domain, and (iii) an intracellular signaling domain comprising an activation domain and optionally a co-stimulatory domain;
wherein expression of the second nucleic acid that encodes a CAR is inducible in a cell upon binding of the synNotch receptor encoded by said first nucleic acid to (a.) PSMA or to (b.) the other antigen which is a marker of senescence in prostate cancer cells.

A vector comprising a nucleic acid, or a pair of nucleic acids according to the invention also makes part of the invention.

The invention further relates to an immunoresponsive cell that comprises a nucleic acid, a pair nucleic acids, or a vector according to the invention.

The invention further relates to a nucleic acid, pair nucleic acids, vector, or immunoresponsive cell according to the invention for use as a medicament or for treating prostate cancer.

### DETAILED DESCRIPTION

### Definitions

“Prostate-specific membrane antigen” or “PSMA” is a type II membrane protein expressed in all forms of prostate tissue. A reference sequence of human PSMA is available in Uniprot database, under accession number Q04609 (entry version 222 of 3 May 2023).

“Nectin2” denotes a type I membrane glycoprotein with two Ig-like C2-type domains and an Ig-like V-type domain, which is a plasma membrane component of adherens junctions. A reference sequence of human Nectin2 is available in Uniprot database, under accession number Q92692 (entry version 217 of 3 May 2023). Nectin2 is also named Poliovirus Receptor-Related Protein-2, Poliovirus Receptor-Like 2, CD112, or PRR-2.

An “immunoresponsive cell” is a T cell, a Natural Killer (NK) cell, a CD8⁺ T cell, a cytotoxic T lymphocyte (CTL), a human embryonic stem cell, or a pluripotent stem cell from which lymphoid cells may be differentiated. Preferably the immunoresponsive cell is a T cell or NK cell.

A “chimeric antigen receptor” or “CAR” refers to an engineered receptor that is expressed on a T cell or any other effector cell type capable of cell-mediated cytotoxicity. The CAR includes an antigen-binding domain that is specific for a ligand or receptor. The CAR usually also includes a transmembrane domain, an intracellular domain and a signaling domain.

A “synthetic Notch receptor” or “synNotch receptor” denotes a Notch receptor engineered to generate a novel cell-cell contact signaling pathway. In synNotch, the extra-cellular domain that binds to Notch ligand and an intracellular transcriptional regulatory domain that is released by ligand-induced cleavage are replaced by a ligand biding domain of desired specificity and an orthogonal transcription factor (Morsut et al. Cell, 2016, 164, 780-791).

### CAR, or CAR/synNotch Receptor Encoding Nucleic Acids and Vectors

In a first aspect, at least one nucleic acid is provided that encode(s) two types of chimeric antigen receptors (CARs), one targeting PSMA and the other targeting an antigen other than PSMA, which is a marker of senescence in prostate cancer cells.

According to an embodiment, it is provided a nucleic acid that encodes:
a. a first chimeric antigen receptor (CAR) that comprises (i) an antigen binding domain that binds prostate-specific membrane antigen (PSMA), (ii) a transmembrane domain, and (iii) an intracellular signaling domain comprising an activation domain, and a second CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain, and a second CAR that comprises (i) an antigen binding domain that binds PSMA, (ii) a transmembrane domain, and (iii) an intracellular signaling domain comprising a co-stimulatory domain.

Said nucleic acid is designed so that said first and second CARs are co-expressed in a cell. In an embodiment, the nucleic acid may be a bi-cistronic nucleic acid. In another embodiment, the sequences encoding said first and second CARs can be concatenated into a single transgene wherein the CARs' ORF are linked by a Foot-and-mouth disease virus 2A or ‘2A-like’ sequence (FMD-2A).

According to another embodiment, it is provided a pair of nucleic acids, comprising:
a. a first nucleic acid encodes a CAR that comprises (i) an antigen binding domain that binds prostate-specific membrane antigen (PSMA), (ii) a transmembrane domain, and (iii) an intracellular signaling domain comprising an activation domain, and a second nucleic acid encodes a CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first nucleic acid encodes a CAR that comprises (i) an antigen binding domain that binds an antigen, other than PSMA, which is a marker of senescence in prostate cancer cells, (ii) a transmembrane domain, and (iii) an intracellular signaling domain comprising an activation domain, and a second nucleic acid encodes a CAR that comprises (i) an antigen binding domain that binds PSMA, (ii) a transmembrane domain, and (iii) an intracellular signaling domain comprising a co-stimulatory domain.

These CAR designs ensure that successful immune cell activation requires both binding to PSMA and to the other marker of senescence in prostate cancer cells, hence triggering signaling through the intracellular signaling domain comprising an activation domain, as well as through the intracellular signaling domain comprising a co-stimulatory domain. The activation domain is primarily responsible for activation of T cell and induction of cytolytic response. The co-stimulatory domain(s) contribute functionally to the in vivo persistence of CAR-T cells.

The activation domain and/or costimulatory domain is an intracellular domain from one or more protein selected from the group consisting of OX40, CD2, CD27, CD28, CD3 zeta, ICAM-1, LFA-1 (CD1 la/CD18), ICOS (CD278), and 4-1 BB (CD137).

Preferably, said intracellular signaling domain comprising an activation domain comprises an activation domain of CD3 zeta.

Preferably, said intracellular signaling domain comprising a co-stimulatory domain comprises a co-stimulatory domain of 4-1BB (CD137) and/or CD28.

In some embodiments, the antigen binding domain of one or both CARs is selected from the group consisting of an antibody, a Fv, a scFv, a Fab, or another antibody fragment. Preferably the antigen binding domain of one or both CARs is a scFv. The ScFv may comprise or consist of a VH domain linked to a VL domain by a (G4S) 3 linker.

In some embodiments, the transmembrane domain is a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. Preferably, the transmembrane domain is the transmembrane domain of CD8 or CD28.

In some embodiments, the antigen binding domain of one or both CARs is connected to the transmembrane domain by a hinge domain. Preferably, the hinge domain is a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. Still preferably, the hinge domain derive from the same protein as the transmembrane domain. Preferably, the hinge domain is the hinge domain of CD8 or CD28.

In an embodiment, the nucleic acid comprises a nucleotide sequence encoding a combo CAR polypeptide comprising (i) a VH region of an antibody directed to one of PSMA and the other antigen marker of senescence, (ii) a linker, such as a (G4S) 3 linker, (iii) a VL region of said antibody directed to one of PSMA and the other antigen marker of senescence, (iv) a CD8 hinge domain, (v) a CD8 transmembrane domain, (vi) an intracellular domain of CD3 zeta, (vii) a FMD-2A peptide, (viii)) a VH region of an antibody directed to the other of PSMA and the other antigen marker of senescence, (ix) a (G4S) 3 linker, (x) a VL region of said antibody directed to the other of PSMA and the other antigen marker of senescence, (xi) a CD8a Stalk domain, (xii) a CD28 transmembrane domain, and (vi) an intracellular domain of 4-1BB.

In an embodiment, the nucleic acid comprises or consists of sequence SEQ ID NO:5, In an embodiment, the nucleic acid encodes the polypeptide that comprises or consists of sequence SEQ ID NO:6.

In an embodiment, the pair of nucleic acids comprises:
a. a first nucleic acid encoding a CAR comprising (i) a VH region of an antibody directed to one of PSMA and the other antigen marker of senescence, (ii) a linker, such as a (G4S) 3 linker, (iii) a VL region of said antibody directed to one of PSMA and the other antigen marker of senescence, (iv) a CD8a Stalk domain, (v) a CD28 transmembrane domain, (vi) an intracellular domain of 4-1BB, and (vii) an intracellular domain of CD3 zeta; and
b. a second nucleic acid encoding a CAR comprising (i) a VH region of an antibody directed to the other one of PSMA and the other antigen marker of senescence, (ii) a linker, such as a (G4S) 3 linker, (iii) a VL region of said antibody directed to the other one of PSMA and the other antigen marker of senescence, (iv) a CD8 hinge domain, (v) a CD8 transmembrane domain, (vi) an intracellular domain of 4-1BB, and (vii) an intracellular domain of CD3 zeta.

In an embodiment, the pair of nucleic acids comprises a nucleic acid that comprises or consists of sequence SEQ ID NO:1, or a sequence at least 85%, 90% or 95% identical thereto that encodes a CAR directed against PSMA. In an embodiment, the pair of nucleic acids comprises a nucleic acid that encodes a CAR directed against Nectin2. In an embodiment said CAR directed against Nectin2 binds to a cancer-exposed epitope of Nectin2. The CAR directed against Nectin2 therefore discriminates cancerous Nectin2-expressing human cells from non-malignant Nectin2-expressing human cells. In an embodiment said CAR directed against Nectin2 binds to an epitope comprising or consisting of sequence SEQ ID NO: 7. In an embodiment, the pair of nucleic acids comprises a nucleic acid that comprises or consists of sequence SEQ ID NO:3, or a sequence at least 85%, 90% or 95% identical thereto that encodes a CAR directed against Nectin2. In an embodiment, the pair of nucleic acids comprises a first nucleic acid that comprises or consists of sequence SEQ ID NO:1, or a sequence at least 85%, 90% or 95% identical thereto that encodes a CAR directed against PSMA, and a second nucleic acid that comprises or consists of sequence SEQ ID NO:3, or a sequence at least 85%, 90% or 95% identical thereto that encodes a CAR directed against Nectin2.

In an embodiment, the pair of nucleic acids comprises a nucleic acid that encodes a CAR comprising or consisting of sequence SEQ ID NO:2, or a sequence at least 85%, 90% or 95% identical thereto that binds to PSMA. In an embodiment, the pair of nucleic acids comprises a nucleic acid that encodes a CAR comprising or consisting of sequence SEQ ID NO: 4, or a sequence at least 85%, 90% or 95% identical thereto that binds to Nectin2. In an embodiment, the pair of nucleic acids comprises a first nucleic acid that encodes a CAR comprising or consisting of sequence SEQ ID NO:2, or a sequence at least 85%, 90% or 95% identical thereto that binds to PSMA, and a second nucleic acid that encodes a CAR comprising or consisting of sequence SEQ ID NO:4, or a sequence at least 85%, 90% or 95% identical thereto that binds to PSMA.

As used herein, the percentage of identity is calculated after pairwise global sequence alignment with Smith-Waterman algorithm, for instance using EMBOSS Needle with default settings (matrix BLOSUM62, gap open 10, gap extend 0.5, end gap penalty false, end gap open 10, end gap extend 0.5).

In a second aspect, a pair of nucleic acids is provided that encode a synthetic Notch (synNotch) receptor and a CAR, one targeting PSMA and the other targeting an antigen other than PSMA, which is a marker of senescence in prostate cancer cells.

The pair of nucleic acids thus comprises:
a. a first nucleic acid that encodes a synthetic Notch (synNotch) receptor comprising an antigen binding domain that binds prostate-specific membrane antigen (PSMA); and a second nucleic acid that encodes a chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain and optionally a co-stimulatory domain; or
b. a first nucleic acid that encodes a synthetic Notch (synNotch) receptor comprising an antigen binding domain that binds an antigen, other than PSMA, which is a marker of senescence in prostate cancer cells; and a second nucleic acid that encodes a chimeric antigen receptor (CAR) that comprises (i) an antigen binding domain that binds PSMA, (ii) a transmembrane domain, and (iii) an intracellular signaling domain comprising an activation domain and optionally a co-stimulatory domain;
wherein expression of the second nucleic acid that encodes a CAR is inducible in a cell upon binding of the synNotch receptor encoded by said first nucleic acid to (a.) PSMA or to (b.) the other antigen which is a marker of senescence in prostate cancer cells.

In some embodiments, the antigen binding domain of the CAR is connected to the transmembrane domain by a hinge domain. In some embodiments, the intracellular signaling domain of the CAR comprises an activation domain and a co-stimulatory domain. The antigen binding domain, transmembrane domain, intracellular signaling domain comprising an activation domain and optionally a co-stimulatory domain are as defined above.

In an embodiment, the synNotch receptor comprises said antigen binding domain that binds PSMA or the antigen, other than PSMA, which is a marker of senescence in prostate cancer cells, fused to a Notch regulatory region, and a cleavable intracellular domain comprising a transcription factor. In said embodiment, the nucleic acid that encodes the CAR is under the control of a promoter that requires activation by the transcription factor comprised in the synNotch receptor. An example of suitable pair of transcription factor/promoter include TetR-VP64 with TRE3GS inducible promoter. The synNotch receptor technology is further described in Morsut et al., 2016, Cell 164, 780-791, and Roybal et al., 2016, Cell 164, 770-779, that are both herein incorporated by reference.

Preferably, in said first and second aspects, the antigen other than PSMA, which is a marker of senescence in prostate cancer cells is a cell membrane antigen.

Cellular senescence is an irreversible cell growth arrest usually involving the p53 and the p16INKA tumor suppressors. Markers of senescence in prostate cancer cells include PSMA, and Nectin 2. Accordingly, preferably the antigen other than PSMA, which is a marker of senescence in prostate cancer cells is Nectin 2.

In some embodiments, the CAR directed against Nectin2, or antigen binding domain of the synNotch receptor, binds to a cancer-exposed epitope of Nectin2. The CAR or synNotch receptor therefore discriminates cancerous Nectin2-expressing human cells from non-malignant Nectin2-expressing human cells. In some embodiments, said CAR directed against Nectin2, or synNotch receptor, binds to an epitope comprising or consisting of sequence SEQ ID NO: 7.

In an embodiment, the nucleic acid that encodes a CAR comprises a nucleic acid that comprises or consists of sequence SEQ ID NO:3, or a sequence at least 85%, 90% or 95% identical thereto that encodes a CAR directed against Nectin2.

The invention further relates to a vector comprising a nucleic acid or a pair of nucleic acids as described above.

In some embodiments the vector is selected from the group consisting of a DNA, a RNA, a plasmid, a lentivirus vector, an adenoviral vector, or a retrovirus vector. Preferably the vector is a lentivirus vector.

### Immunoresponsive Cell

The invention also relates to an immunoresponsive cell that comprises a nucleic acid, a pair nucleic acids, or a vector as described above.

In a first aspect, the immunoresponsive cell comprises:
a. a first CAR that comprises (i) an antigen binding domain that binds prostate-specific membrane antigen (PSMA), (ii) a transmembrane domain, and (iii) an intracellular signaling domain comprising an activation domain, and a second CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first CAR that comprises (i) an antigen binding domain that binds an antigen, other than PSMA, which is a marker of senescence in prostate cancer cells, (ii) a transmembrane domain, and (iii) an intracellular signaling domain comprising an activation domain, and a second CAR that comprises (i) an antigen binding domain that binds PSMA, (ii) a transmembrane domain, and (iii) an intracellular signaling domain comprising a co-stimulatory domain.

In an embodiment, said immunoresponsive cell expresses at its membrane two types of chimeric antigen receptors (CARs), one targeting PSMA and the other targeting an antigen other than PSMA, which is a marker of senescence in prostate cancer cells, as disclosed above.

In an embodiment, said immunoresponsive cell is activated only upon binding to both PSMA and said antigen, other than PSMA, which is a marker of senescence in prostate cancer cells.

In a second aspect, the immunoresponsive comprises:
a. a synthetic Notch (synNotch) receptor comprising an antigen binding domain that binds prostate-specific membrane antigen (PSMA); and a nucleic acid that encodes a chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain and optionally a co-stimulatory domain; or
b. a synNotch receptor comprising an antigen binding domain that binds an antigen, other than PSMA, which is a marker of senescence in prostate cancer cells; and a nucleic acid that encodes a chimeric antigen receptor (CAR) that comprises (i) an antigen binding domain that binds PSMA, (ii) a transmembrane domain, and (iii) an intracellular signaling domain comprising an activation domain and optionally a co-stimulatory domain;
wherein expression of the nucleic acid that encodes a CAR is inducible in a cell upon binding of the synNotch receptor encoded by said first nucleic acid to (a.) PSMA or to (b.) the other antigen which is a marker of senescence in prostate cancer cells.

In an embodiment, the immunoresponsive cell expresses at its membrane a synNotch receptor targeting one of PSMA and the other targeting an antigen other than PSMA, which is a marker of senescence in prostate cancer cells.

In an embodiment, the immunoresponsive cell further expresses at its membrane a CAR targeting the other one of PSMA and the other targeting an antigen other than PSMA, which is a marker of senescence in prostate cancer cells. Co-expression of the synNotch receptor and CAR in the immunoresponsive cell is to occur upon binding of the synNotch receptor to its target which triggers induction of CAR expression in the immunoresponsive cell.

In an embodiment, said immunoresponsive cell is activated only upon binding to both PSMA and said antigen, other than PSMA, which is a marker of senescence in prostate cancer cells.

Altogether, said immunoresponsive cell may express the synNotch receptor, the synNotch receptor and CAR, or the pair of CARs at its membrane.

Preferably, in said first and second aspects, the antigen other than PSMA, which is a marker of senescence in prostate cancer cells is a cell membrane antigen.

Cellular senescence is an irreversible cell growth arrest usually involving the p53 and the p16INKA tumor suppressors. Markers of senescence in prostate cancer cells include PSMA, and Nectin 2. Accordingly, preferably the antigen other than PSMA, which is a marker of senescence in prostate cancer cells is Nectin 2.

### Therapeutic Uses

The nucleic acid, pair of nucleic acids, vector, or immunoresponsive cell according to the invention may be used in therapeutic methods.

In particular, the invention further relates to nucleic acid, pair of nucleic acids, vector, or immunoresponsive cell as described above for use as a medicament.

The invention further relates to a method of treatment which comprises administering a nucleic acid, pair of nucleic acids, vector, or immunoresponsive cell as described above to a subject in need thereof.

In an embodiment the invention relates to nucleic acid, pair of nucleic acids, vector, or immunoresponsive cell as described above for use for treating prostate cancer, in particular is a castration-resistant prostate cancer, or metastatic castration-resistant prostate cancer.

Accordingly, a method for treating prostate cancer, in particular is a castration-resistant prostate cancer, or metastatic castration-resistant prostate cancer, is provided which comprises administering a nucleic acid, pair of nucleic acids, vector, or immunoresponsive cell as described above to a subject in need thereof.

The subject may be a mammal, such as a primate (e.g. human, monkey), a rodent (e.g. rat, mouse), a canine (e.g. a dog), or a feline (e.g. a cat). Preferably, the subject is a human.

In some embodiments at least one prostate cancer therapeutic agent is used in combination with the nucleic acid, pair of nucleic acids, vector, or immunoresponsive cell. Said prostate cancer therapeutic agent may be selected from the group consisting of chemotherapeutic agents (such as Docetaxel, or Enzalutamide), and immune checkpoint inhibitors (such as anti-PD1 or anti-TIGIT antibodies).

The invention is further illustrated by the following figures and examples.

### FIGURES

FIG. 1. Nectin2-PVRIG axis is upregulated in PCa. (A) mRNA expression level of different immune checkpoint expressed by epithelial and immune cells from bulk-RNA data. (B) Schematic representation of the Nectin2 and PVR interaction with receptor expressed by T and NK cells. (C) Gene expression level on human prostate cancer samples. Among Nectin-like molecules, Nectin2 is the most abundant one in epithelial cells. On the other hand, PVRIG is the most expressed receptor in immune cells.

FIG. 2. Nectin2 is upregulated in Pten-loss-induced cellular senescence (PICS). (A) Experimental design and Volcano plot of proteins differentially present between FDG+ and FDG− epithelial cells. (B) Results of western blot analysis in wild type and pten−/− prostate samples and quantification of the fold change compared to wild type of Nectin2 and p21. (C) Real time PCR data of Nectin2 and p16 mRNA expression level in FDG+ and FDG-sorted epithelial prostate tumor cells. (D) Nectin2 expression by RNA-seq data on wild type, pten−/− and pten−/−p53−/− mice. (E) PTEN null mice were treated with Docetaxel 10 mg/Kg once a week for four weeks. After 4 weeks, the prostate was collected, and senescence induction was quantified through beta-galactosidase assay on OCT embedded tissue. (F) Anterior lobe size was significantly reduced in treated mice compared to untreated one and (G) real time PCR on prostate samples confirmed Nectin2 upregulation.

FIG. 3. Therapy-induced senescence (TIS) further enhances the level of Nectin2 in PCa. (A) Senescence induction in PC3, 22RV1 and LNCaP human prostate cancer cell lines using Palbociclib and Docetaxel. Treatments were added in the medium for three days, then the medium was changed and after three days cells were seeded for beta-galactosidase assay. (B) Nectin2 expression in human prostate cancer cell lines after senescence induction using Palbociclib or Docetaxel by real-time and (C) flow cytometry. (D) Senescence induction upon therapy induced senescence measured through FDG staining in 22rv1 cells and (E) mean fluorescence intensity (MFI) of Nectin2 in untreated and treated cells. (F) MFI of Nectin2 cells in FDG− and FDG+ population and percentage of FDG− and FDG+ LNCAP cells untreated or cultured in androgen deprivation (ADT). (G) Results of beta-galactosidase assay in LNCAP upon senescence induction using androgen deprivation and (H) Nectin2 mRNA level.

FIG. 4. Nectin2 and PSMA expression correlates in PCa. (A) Correlation matrix showing Pearson coefficients in human RNA seq samples. (B) Scatter plot representing correlation between Nectin2 and FOLH1, and PVR and FOLH1. (C) Percentage of Nectin2+PSMA−, Nectin2+PSMA+, Nectin2−PSMA+ and Nectin2−PSMA− in tumor cells. (D) Comparison of the percentage of Nectin2+PSMA−, Nectin2+PSMA+, Nectin2-PSMA+ and Nectin2−PSMA− in tumor cells in castration-sensitive and castration-resistance prostate cancer patients. (E) Western blot on patient-derived organoid shows a correlation between Nectin2 and PSMA. (F) PSMA protein expression level by western blot on human prostate cancer cell lines upon TIS.

FIG. 5. Anti-PSMA and anti-Nectin2 combinatorial CAR T cells specifically lyse PSMA+/Nectin2+ and PSMA+/Nectin2− human PCa cell lines. (A) Target antigen expression on human PCa cell lines as assessed via flow cytometry. (B) CAR design for second generation monospecific anti-PSMA and anti-Nectin2 CAR constructs. (C) CAR design for combinatorial CARs targeting PSMA and Nectin2. (D) CAR expression on T cells versus non-transduced (NTD) as assessed by flow cytometry. (F-G) CombiCAR T cells specifically lyse PSMA+/Nectin2+ and PSMA+/Nectin2− human PCa cells in 92-hr fluorescence-based lysis assay. (E) ELISA determination of the secretion of IFNg by CAR T cells in co-culture with tumor cells as described in (F-G).

FIG. 6. Anti-Nectin2 scFv-Fc (725v5) is binds to a cancer-exposed epitope of Nectin2. The anti-Nectin2 scFv-Fc (725v5) selectively binds to Nectin 2 when expressed by tumor cells, by contrast with a commercial anti-Nectin2 monoclonal antibody that binds indifferently to normal and tumoral cells expressing Nectin2.

### EXAMPLES

### Example 1: Nectin2 mRNA is Highly Expressed in Prostate Cancer Cells

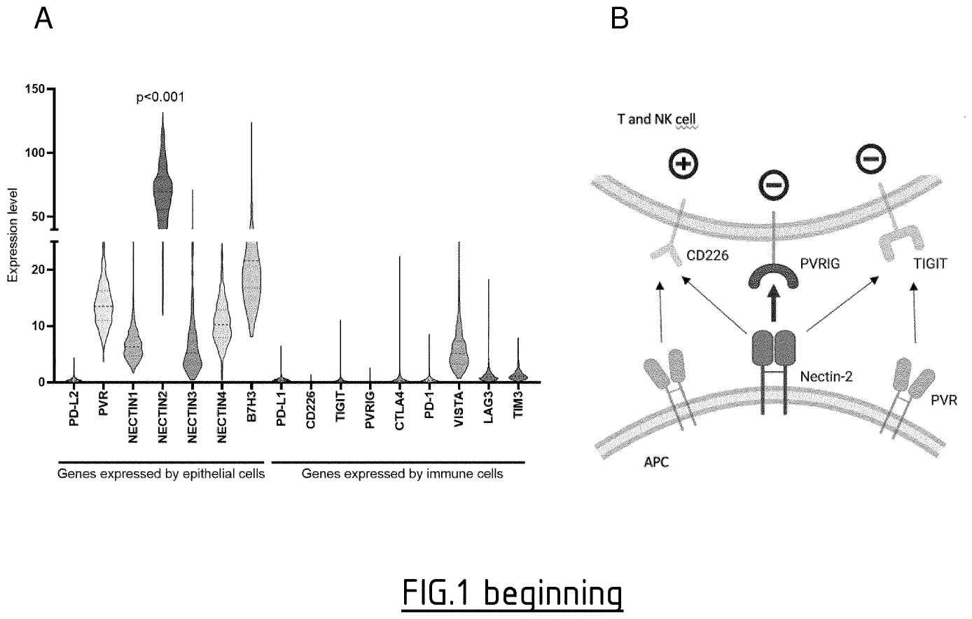

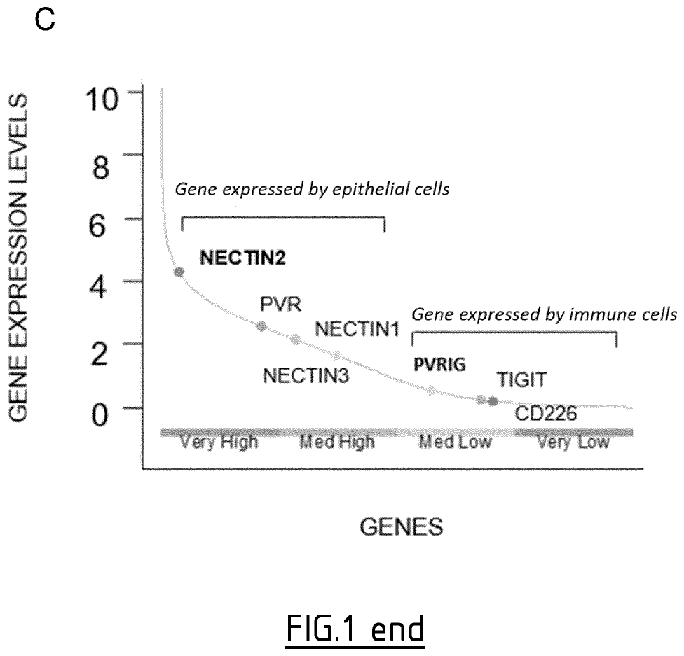

To identify novel targets in prostate cancer we analysed the expression of the most renowned ICs in the publicly available datasets. We compared the mRNA expression levels of genes expressed by epithelial cells and by immune cells using the raw data of the TGCA database from the Human Protein Atlas (https://www.proteinatlas.org/(2022)). Interestingly, as shown in FIG. 1A, we observed a significantly higher expression of Nectin2 in PCa compared to all the other targets, including PD1 and CTLA4.

Nectin2 is a Ca2+-independent cell-cell adhesion molecule, widely expressed on antigen-presenting and tumour cells, whose expression is regulated in tumorigenesis. The role of Nectin2 as an immune checkpoint has already been investigated in breast and ovarian cancer (Oshima, T. et al. Mol. Cancer 12, 60 (2013)). Over-expression of Nectin2 mRNA in prostate cancer tissues was also reported (Oshima, T. et al. Mol. Cancer 12, 60 (2013)). An increase in protein level has been observed in different types of cancer, such as acute myeloid leukaemia, multiple myeloma, gallbladder, and prostate cancer (Sanchez-Correa, B. et al. Immunol. Cell Biol. 90, 109-115 (2012); Casado, J. G. et al. Cancer Immunol. Immunother. Cll 58, 1517-1526 (2009); WO2020144697A1). Although primarily a regulator of cell adhesion, motility and proliferation, Nectin2 is also immunomodulatory (Takai, Y., et al. Nat. Rev. Mol. Cell Biol. 9, 603-615 (2008)). In fact, this membrane protein can either co-stimulate T and NK cells by interacting with CD226 or suppress their response by binding to the inhibiting receptors TIGIT and PVRIG (Zhu, Y. et al. J. Exp. Med. 213, 167-176 (2016)).

It must be emphasized that another Nectin-family member, PVR, is a ligand of the costimulatory receptor CD226 and the co-inhibitory receptor TIGIT (FIG. 1B). On the other hand, the PVR receptor still seems to be the prevailing ligand for TIGIT in this ligand-receptor network because of the feebleness of the Nectin2-TIGIT interaction (Yu, X. et al. Nat. Immunol. 10, 48-57 (2009)).

However, the Nectin2/TIGIT/PVRIG axis in prostate cancer has not yet been investigated.

### Example 2: PVRIG is the Main Nectin2 Interactor in the Prostate Tumor Microenvironment

To better understand the role of Nectin2 in prostate cancer, we then assessed the expression of its ligand/interactors in bulk RNA-seq data from human prostate cancer samples.

We observed a high expression level of Nectin2 and low expression of TIGIT, thus suggesting that PVRIG is the main Nectin2 interactor in the prostate tumor microenvironment (FIG. 1B). Interestingly, by analyzing the single-cell RNA-seq data from PCa biopsies, we verified that Nectin2 is primarily expressed by epithelial cells and endothelial cells (FIG. 1C). These data were obtained starting from 13 tissue samples from 12 primary tumors and 1 lymph node metastasis, and after standard data processing and quality control procedures they got transcriptomic profiles for 36,424 cells (Chen, S. et al. Nat. Cell Biol. 23, 87-98 (2021)). However, this analysis on primary prostate cancer samples is enriched in epithelial cells. Thus, we checked on lethal PC single cell analysis to validate the PVRIG expression by single cell data. The cohort comprised 2,170 cells from 14 patients and 15 fresh biopsies from three common mCRPC metastatic sites (bone, lymph node and liver). PVRIG was confirmed to be expressed by CD4+ T cells, CD8+ T cells and NK cells (Jordan, M. A. & Wilson, L. Nat. Rev. Cancer 4, 253-265 (2004)). In addition, we confirmed the Nectin2 protein expression in the human prostate cancer sample by immunofluorescence (not shown).

### Example 3: Upregulation of Nectin2 is Correlated with Senescence

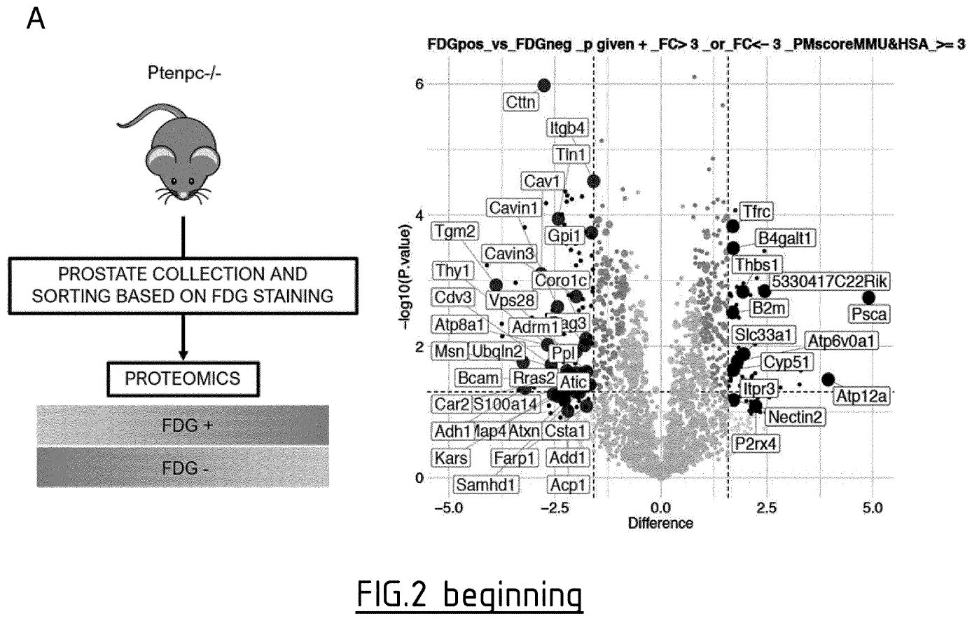

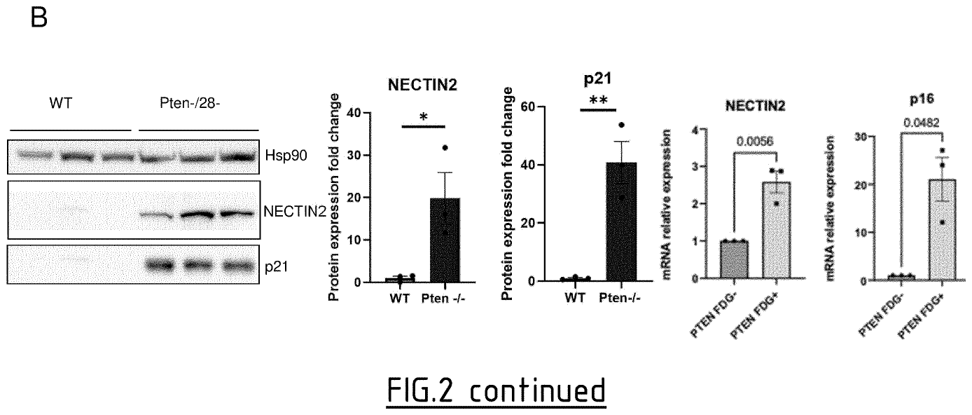

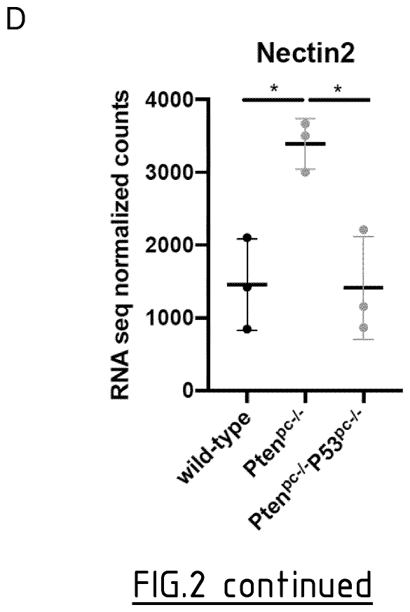

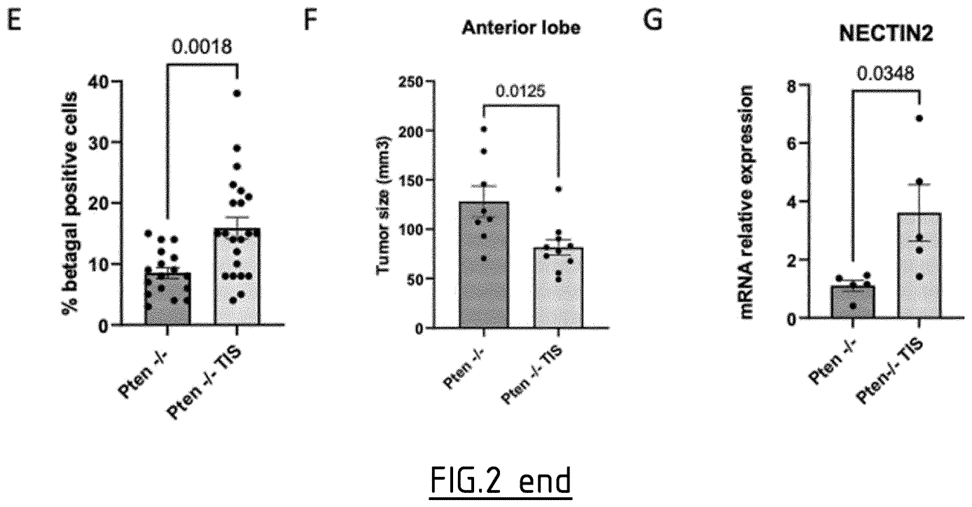

Next, in collaboration with the Proteomic Core Facility of Lausanne at Ecole Polytechnique Fédérale de Lausanne (EPFL), we ran a mass spectrometry and bioinformatic analysis to identify membrane proteins upregulated by the senescent population in Pten/mice. Using this technology, we verified that Nectin2 was among the most upregulated transmembrane proteins expressed by senescent cells (FIG. 2A, right panel). Moreover, the bioinformatic analysis revealed that cells having the highest senescence score were also the ones with the highest Nectin2 abundance.

Using our mouse model of Pten-loss-induced cellular senescence (PICS, Toso et al., Cell Reports 9, 75-89 (2014)), we then demonstrated the correlation at both transcriptional and translation levels between senescence and Nectin2 expression. Western blot confirmed this correlation at translation levels in total tumor (FIG. 2B) and this data was also validated by qRT-PCR on sorted C12−FDG+ and C12−FDG− PTEN null tumor cells (FIG. 2C). Besides Nectin2, we also checked for some senescent markers such as p21 (at translational level) and p16 (at transcriptional level), which were upregulated in the non-proliferative cellular fraction. Moreover, results from RNA-seq confirms the Nectin2 upregulation in pten^{-/-} mice when compared to pten^{-/-};p53^{-/-} and wild-type mice (FIG. 2D).

We then verified in vivo whether the upregulation of Nectin2 correlated or not with therapy-induced senescence (TIS) in our genetically engineered mouse model. Mice were treated with Docetaxel 10 mg/Kg once a week for four weeks and, at the end of the treatment, we performed beta-galactosidase staining to confirm senescence induction (FIG. 2E), in line with a reduction of anterior lobe tumour size (FIG. 2F). Real-time PCR data pointed out a significant Nectin2 upregulation (FIG. 2G).

We validated the expression of our target on human prostate cancer cell lines. To test tumor heterogeneity in terms of mutational burden and genetic alteration, we used human prostate cancer cell lines with both a wild-type and mutated form of PTEN and TP53 gene, and in terms of response to androgen receptor signalling.

To assess whether Nectin2 expression was associated with disease progression and resistance to therapy, we treated human PCa cell lines with two common chemotherapy agents used for PCa management to induce cellular senescence (specifically Docetaxel and Palbociclib.) Docetaxel-based regimens have been shown to improve the patients' symptoms and overall survival in mCRPC patient (Mohler, J. L. et al. J. Natl. Compr. Cancer Netw. JNCCN 17, 479-505 (2019)). Besides, Palbociclib (a CDK4/6 inhibitor) is commonly used for treating metastatic prostate cancer in combination with ADT, and it has already been proven to induce senescence in subtoxic concentrations (ClinicalTrials.gov Identifier: NCT02905318).

It was also observed that the same senescence induction at subtoxic range is true for

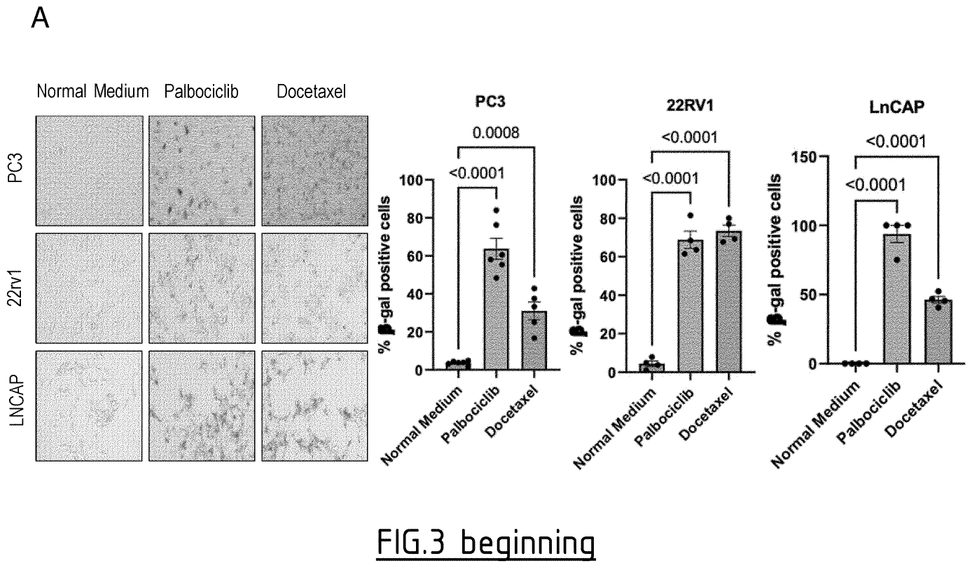

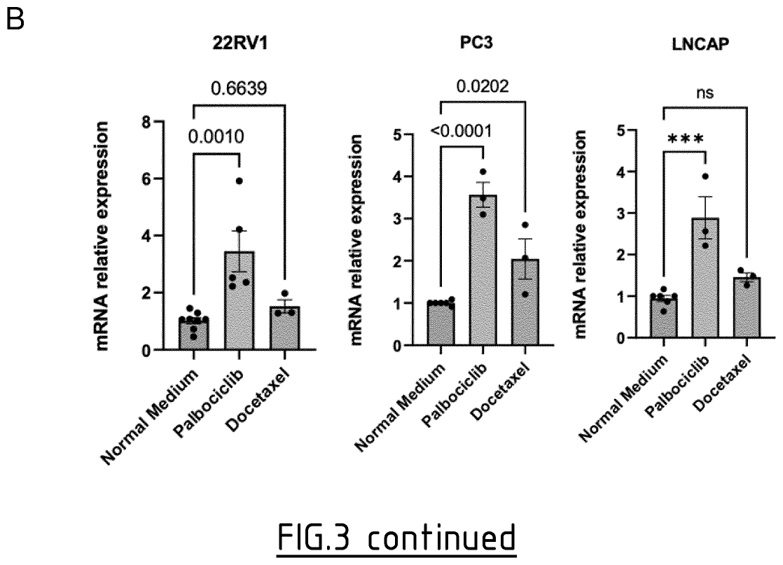

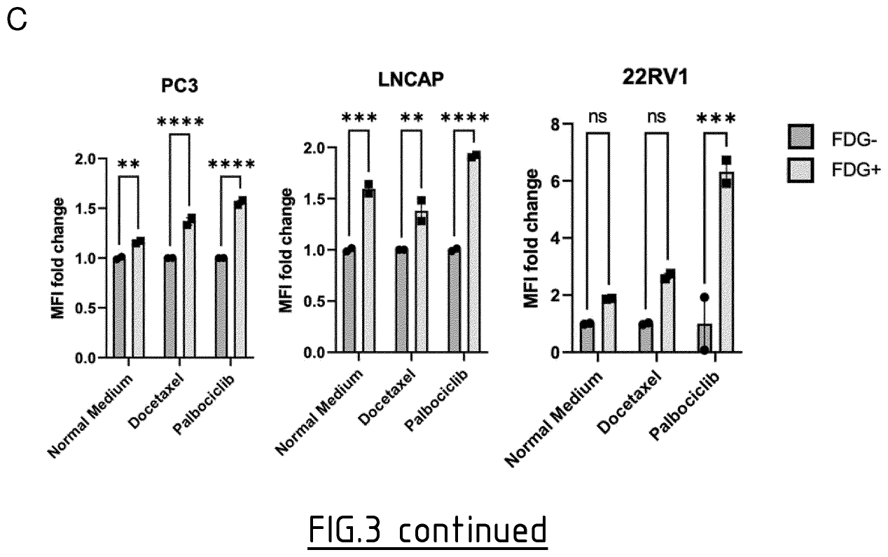

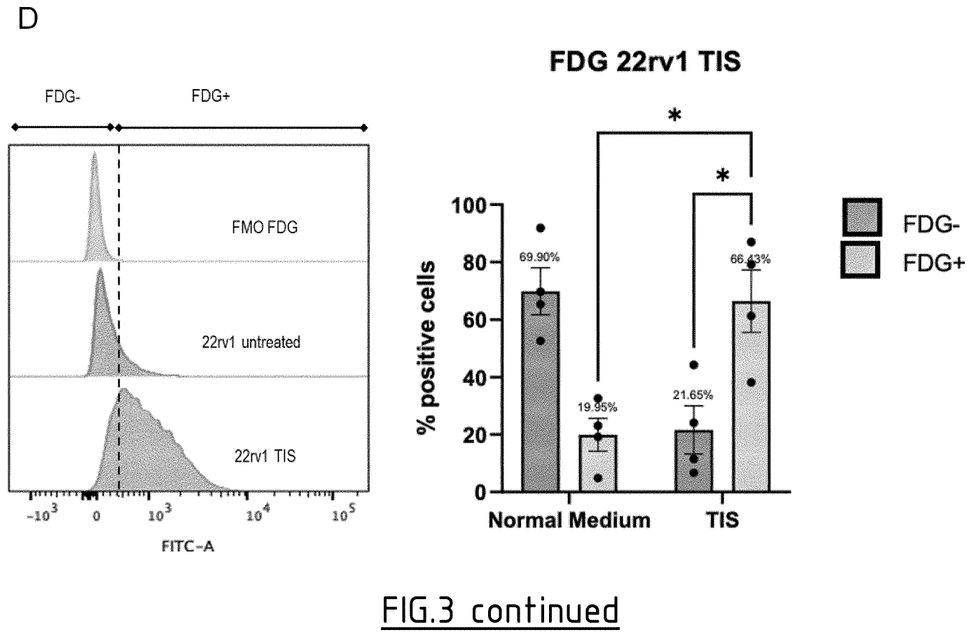

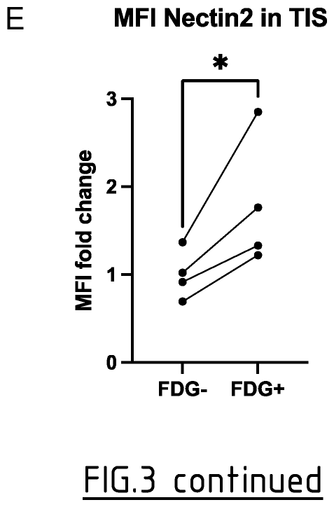

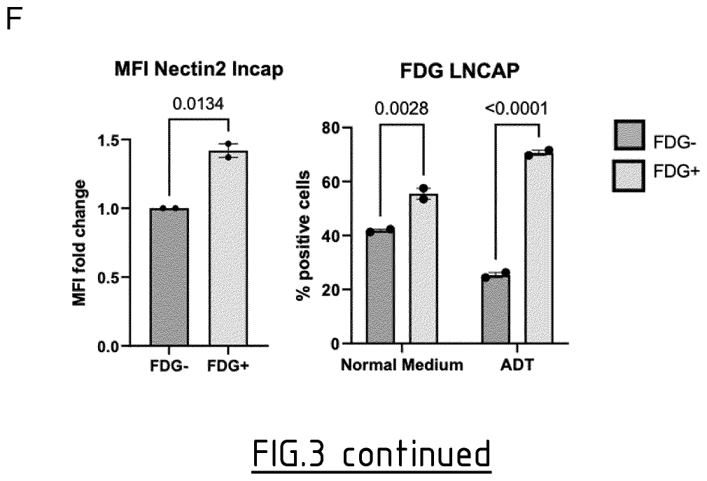

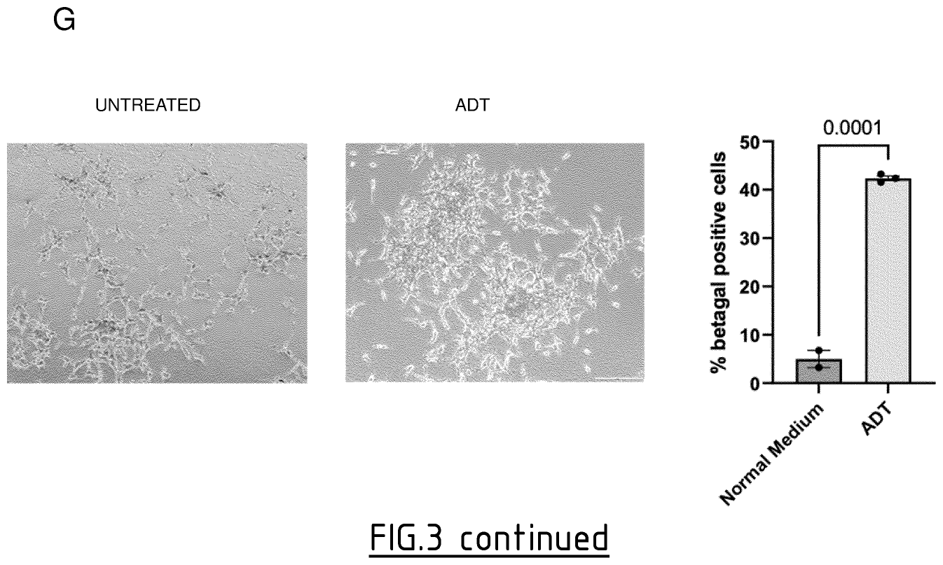

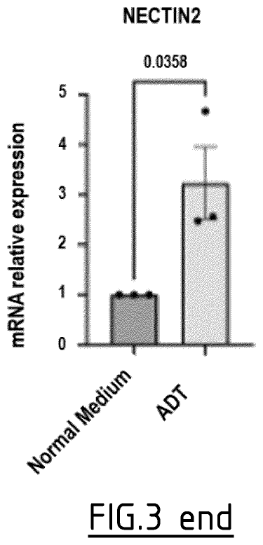

Docetaxel and Palbociclib, and the onset of senescent causes resistance to therapy. Later, the efficacy of senescence induction was evaluated using beta-galactosidase assay (FIG. 3A), and real-time PCR was performed on treated and untreated cells to measure Nectin2 mRNA level (FIG. 3B). Treatment with Palbociclib induced a significant increase in the Nectin2 mRNA level in all the tested cell lines. Induction of Nectin2 mRNA levels after Docetaxel treatment was also substantial compared to the untreated population in PC3 and not significant in 22rv1 and LNCAP cells.

Flow cytometry analysis also confirmed the same results, clearly showing upregulation of Nectin2 at the translational level (FIG. 3C) under all the tested conditions.

Senescence induction upon treatment was also confirmed using FDG staining in 22rv1 (FIG. 3D), as well as Nectin2 upregulation in the treated fraction (FIG. 3E).

The induction of senescence was also achieved through treatment with Enzalutamide, to mimic androgen deprivation therapy. For this purpose, we used LNCaP cells, as they express the androgen receptor and, therefore, they are sensitive to the treatment. As demonstrated through FDG staining (not shown) and beta-galactosidase assay (FIG. 3G), the treatment induced senescence and upregulated Nectin2 at both the protein (FIG. 3F) and mRNA levels (FIG. 3H).

### Example 4: Expression of PSMA Positively Correlates with Nectin2 Expression in Senescent Prostate Cancer Cells

Although the expression of Nectin2 appears cell-specific in the prostate TME, we cannot exclude its expression in other organs.

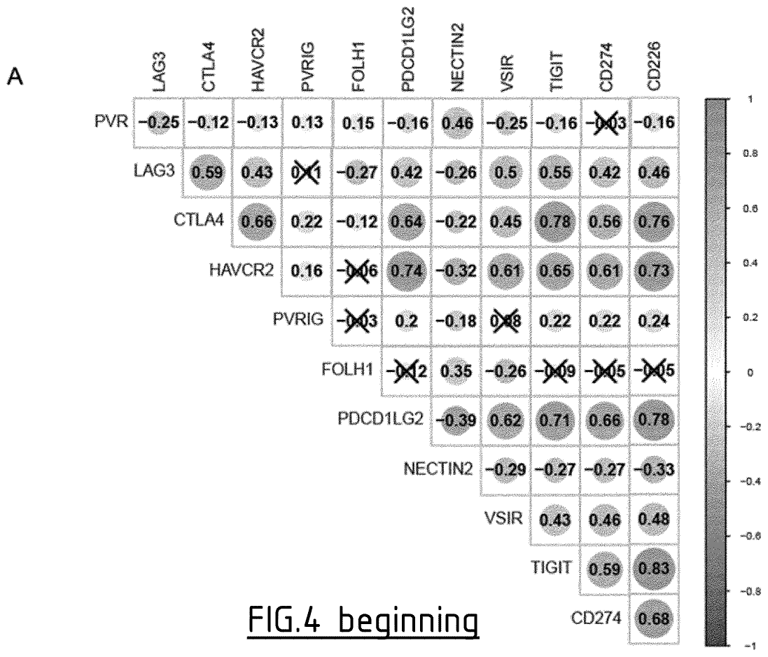

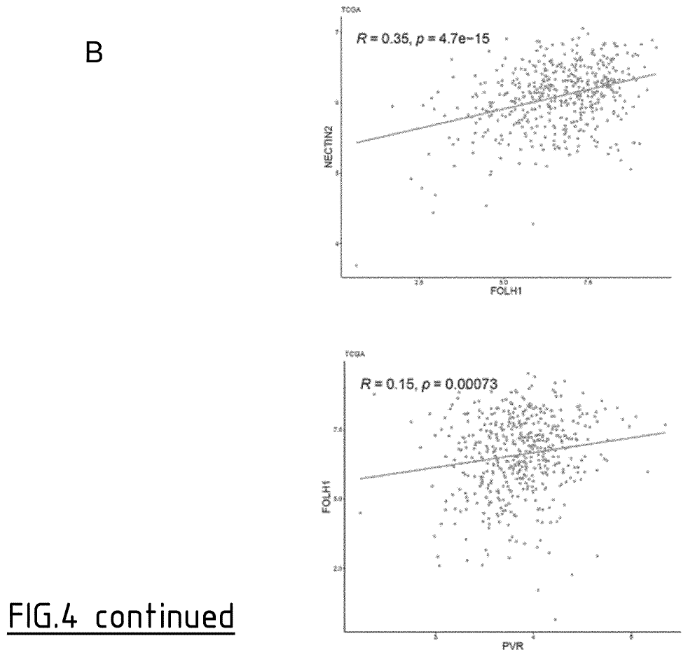

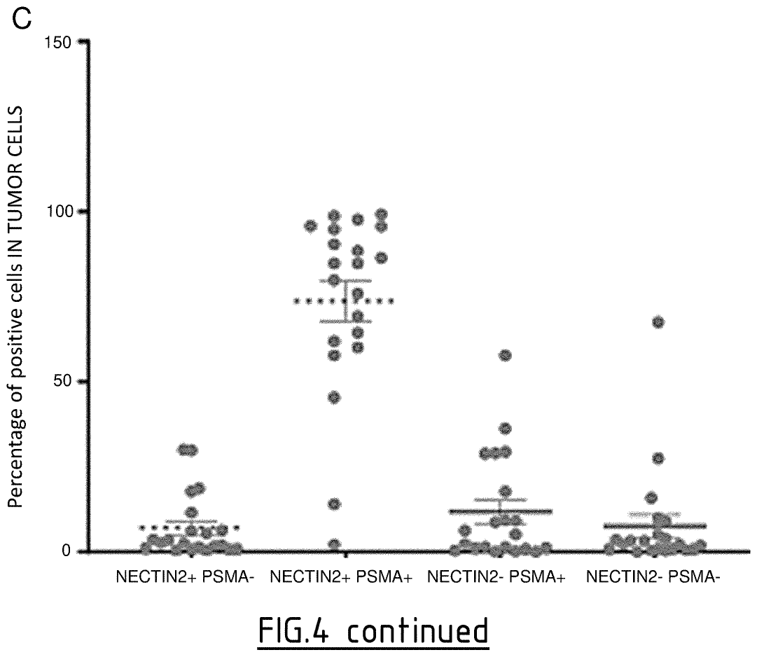

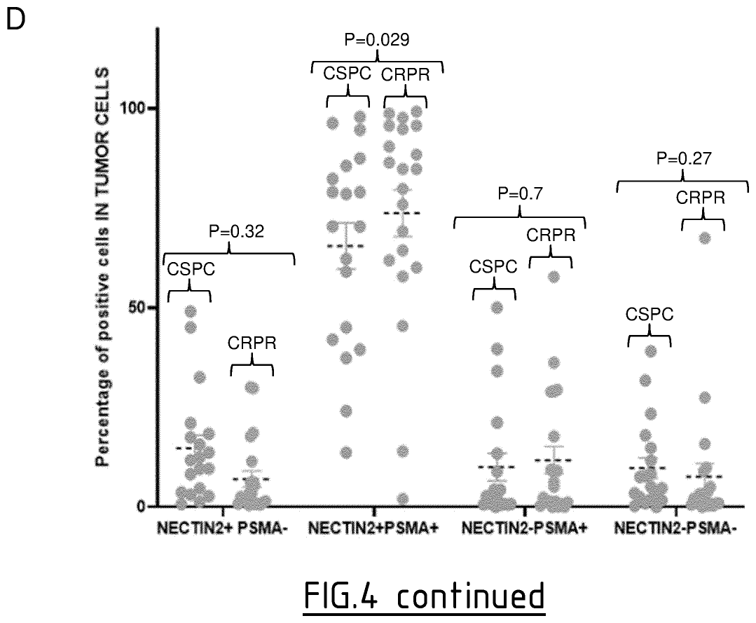

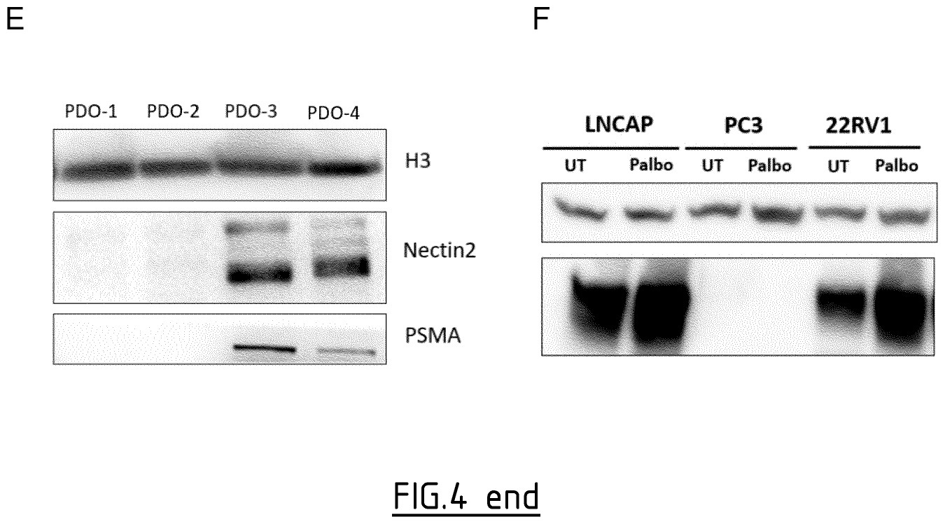

Therefore, we designed a Nectin2-based specific and selective therapeutic strategy by exploiting the prostate-specific membrane antigen (PSMA). To design an effective drug delivery system and immunotherapy, we first checked the correlation of Nectin2 and all the other known immune checkpoints with PSMA. We used the TCGA database and applied a correlation matrix to do that. As shown in FIG. 4A and FIG. 4B, we found that expression of FOLH1 (PSMA) positively correlates only with Nectin2 expression in prostate cancer, which is two times stronger than PVR. Besides, human prostate samples were also analysed by immunofluorescence. In this case, prostate tumour biopsies from normal and tumour tissues were stained using anti-PSMA and anti-Nectin2 primary antibodies. Results of immunofluorescent analysis reveal a higher percentage of epithelial prostate tumour cells co-expressing Nectin2 and PSMA (FIG. 4C). Moreover, samples of castration-sensitive prostate cancer (CSPC) were compared with models of castration-resistant prostate cancer (CRPC) patients, and the percentage of double positive cells was significantly higher in the CRPC ones (FIG. 4D).

Moreover, we performed western blot analysis on Patient-derived human organoids (PDO), and samples characterized by Nectin2 expression were also positive for PSMA (FIG. 4E).

Despite its clinical exploitation, the regulation of PSMA expression in senescent cells has never been investigated. Thus, we checked PSMA protein levels in our senescence models to evaluate the possibility of targeting it in combination with Nectin2. Western blot of human cell lines treated with TIS showed increased PSMA expression in both LNCaP and 22RV1 cells, while no bands were detected in PC3 and since they do not express PSMA (FIG. 4F).

### Example 5: Senolytic Dual CAR-T Cells Targeting Nectin2 and the Prostate-Specific Membrane Antigen (PSMA)

Nowadays, target therapies in prostate cancer are mainly against the prostate-specific membrane antigen (PSMA). The FOLH1 gene encodes PSMA, a type II transmembrane glycoprotein with folate hydrolase and N-acetylated-alpha-linked-acidic dipeptidase activity. PSMA is expressed in normal, benign and malignant prostate tissues, including intraepithelial and metastatic neoplasia (Hupe, M. C. et al. Front. Oncol. 8, 623 (2018); Troyer, J. et al. Int. J. Cancer 62, 552-558 (1995)). However, it has already been shown that PSMA expression is 100- to 1000-fold times higher in prostatic adenocarcinoma compared to benign prostate (Heston, W. D. W. Urology 49, 104-112 (1997)). For this reason, many radiopharmaceutical and diagnostic tools have been designed to target PSMA.

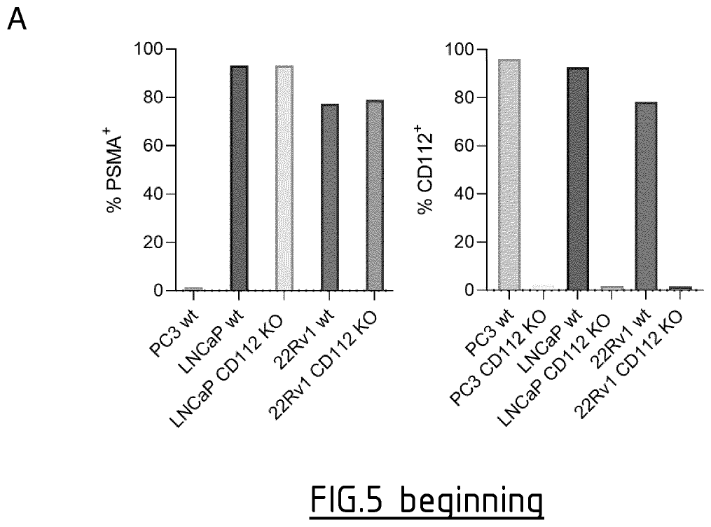

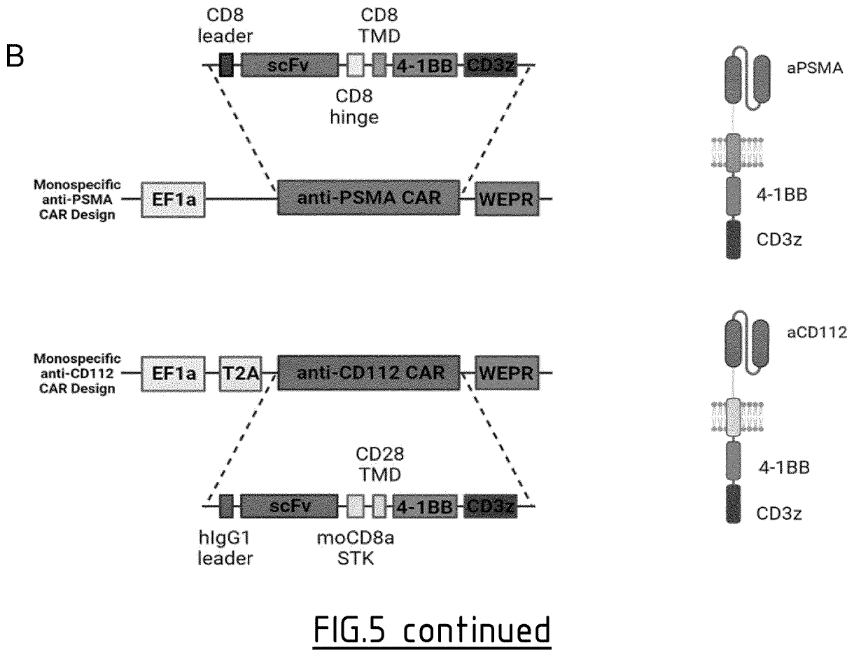

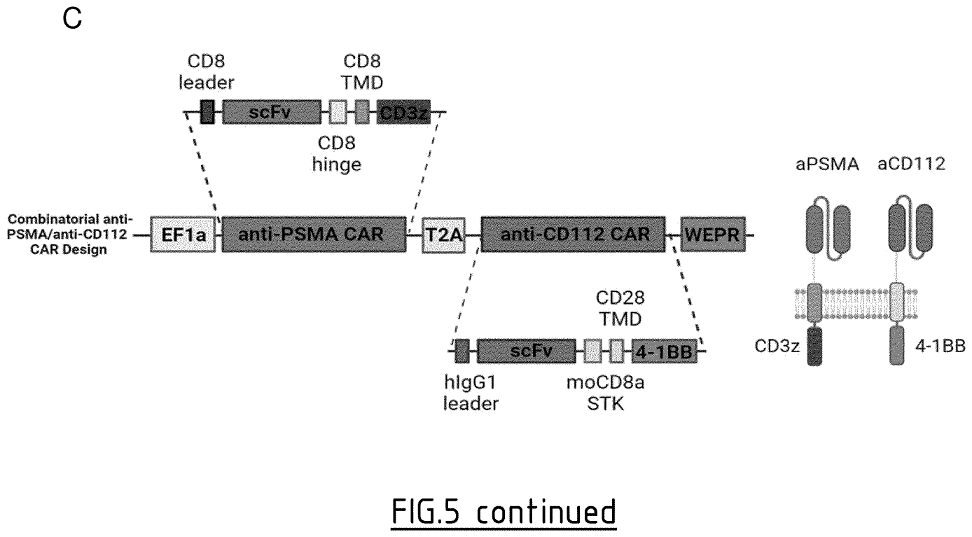

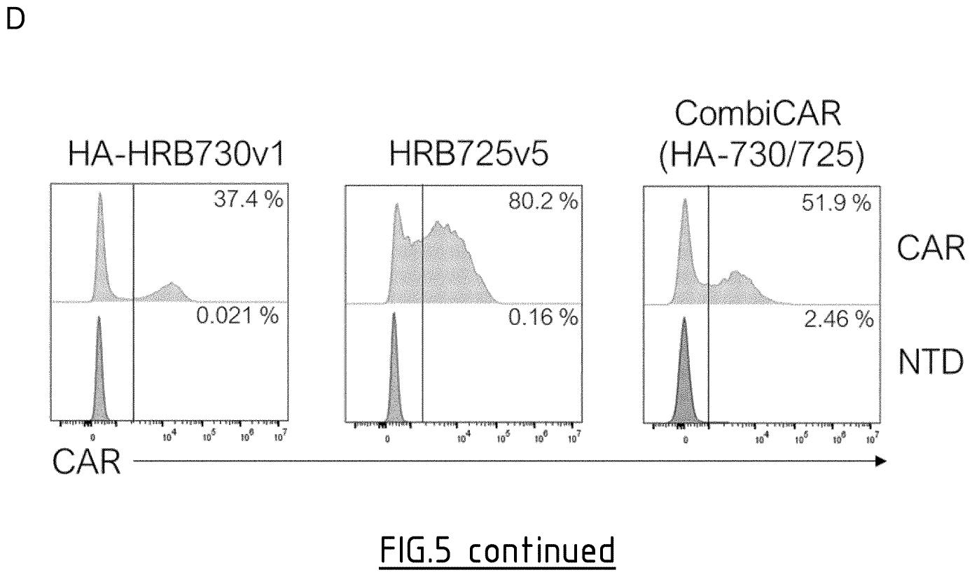

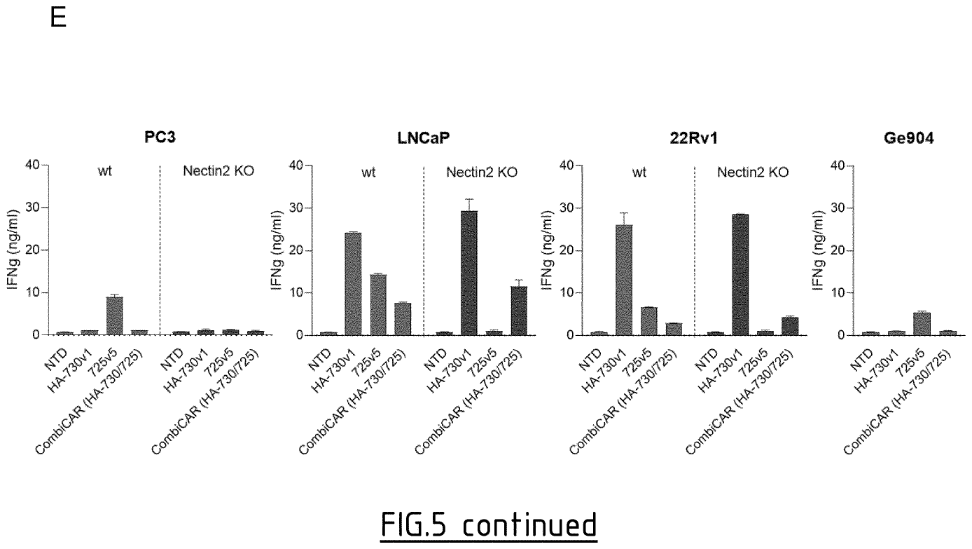

To prevent on-target/off-tumor toxicity with targeting PSMA and Nectin2, we designed a combinatorial CAR T cell approach, a design first described by Kloss et al. to enforce tumor specificity in the absence of tumor-restricted antigens (Kloss, C. et al. Nat. Biotechnol. 31, 71-75 (2013)). Thus, in order to design highly specific and efficient CAR T cells, we screened a phage-display library against PSMA and Nectin2 and generated scFv-Fc constructs with novel anti-PSMA and anti-Nectin2 scFv candidates. To assess their binding specificity to PSMA or Nectin2, we used the PC3 cell line naturally lacking PSMA expression, and generated Nectin2-deficient human PCa cell lines (PC3, LNCaP, 22Rv1) using CRISPR/Cas9 technology. The absence or presence of surface PSMA, as well as the knock-out (KO) of Nectin2 was confirmed via flow cytometry (FIG. 5A). Anti-PSMA (HA-730v1) or anti-Nectin2 (725v5) scFv-Fc constructs were identified to specifically bind to their target antigens in a dose-dependent manner as assessed via flow cytometry staining of PCa cell lines with anti-PSMA or anti-Nectin2 scFv-Fc constructs in different dilutions (1:1, 1:10, 1:500) and an anti-human Fc-AF488 secondary antibody.

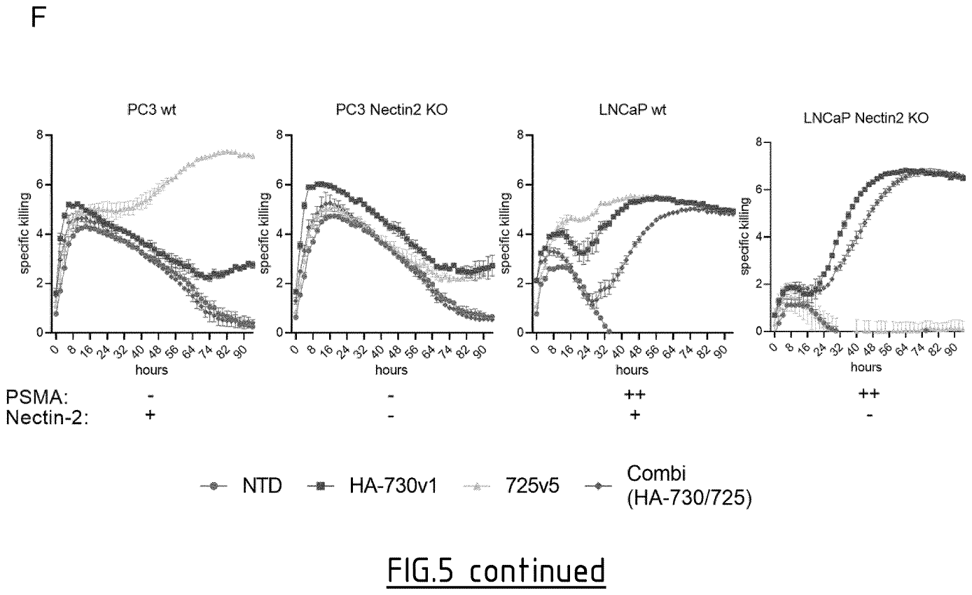

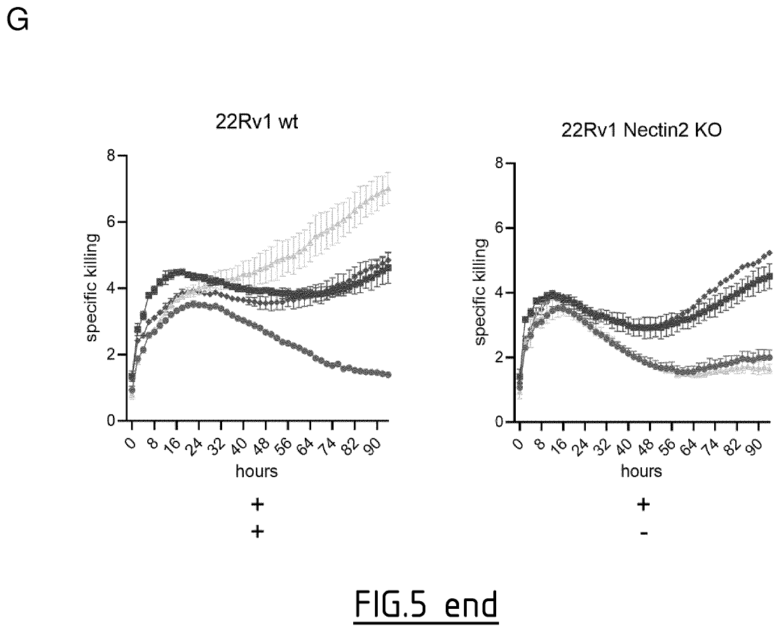

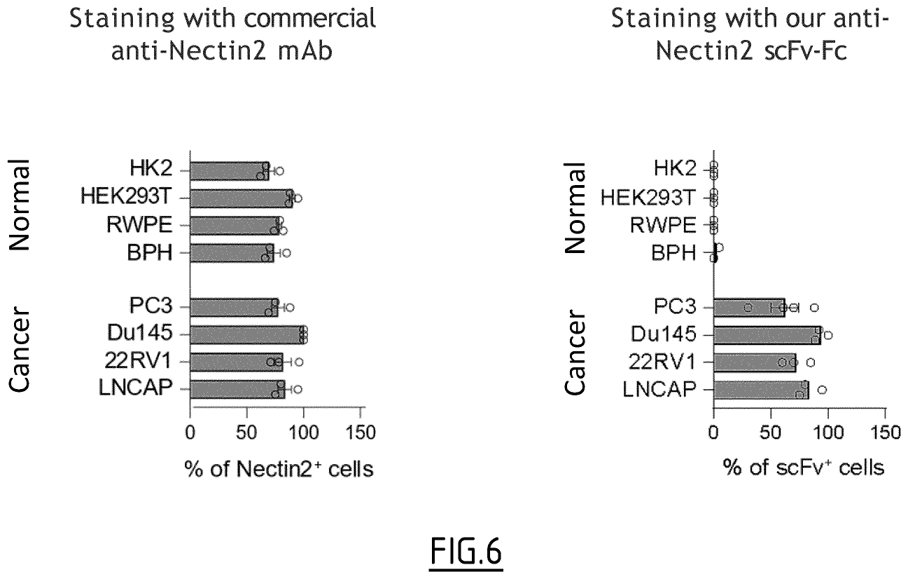

Importantly, the anti-Nectin2 (725v5) scFv-Fc construct was found to be selective of Nectin 2 when expressed on tumor cells, only (FIG. 6). The Nectin2 scFv binds to a cancer-exposed epitope of Nectin2 (comprising or consisting of sequence SEQ ID NO:7), sparing non-malignant Nectin2-expressing human cells.

Subsequently, we designed and generated monospecific anti-PSMA (SEQ ID NO:1-2) or anti-Nectin2 (SEQ ID NO:3-4) CAR constructs (FIG. 5B), as well as a novel CombiCAR construct (SEQ ID NO:5-6) (FIG. 5C). Here, the PSMA-targeting scFv is linked to a transmembrane domain (TMD), and an intracellular CD3z signaling domain, followed by a self-cleaving T2A peptide, and the anti-Nectin2-targeting scFv linked to a distinct TMD and an intracellular 4-1BB co-stimulatory domain. Successful T cell activation requires both the intracellular CD3z signaling, as well as the co-stimulatory signal. Thus, this design ensures that both receptors must simultaneously engage to target Nectin2+ cells in order to trigger downstream signaling leading to T cell activation. After T cell transduction with the respective constructs and CAR T cell expansion, CAR expression was assessed via flow cytometry (FIG. 5D). To determine their anti-tumor killing capacities, CAR T cells were co-cultured with human PCa cell lines for 92h (FIG. 5E). As shown in FIG. 5E, PCa cell lines expressing PSMA are readily lysed by monospecific anti-PSMA CAR T cells, while the PSMA-deficient PCa cell lines PC3 wt and its Nectin2 KO counterpart, as well as the human glioblastoma cell line Ge904, are spared. Similarly, monospecific anti-Nectin2 CAR T cells specifically and efficiently target Nectin2-expressing PCa cell lines. Interestingly, PCa cell lines co-expressing PSMA and Nectin2 are efficiently lysed by CombiCAR T cells, and PSMA-deficient PC3 wt or Ge904 cells are spared. PSMA+/Nectin2− PCa cell lines are lysed by CombiCAR T cells as well, as the PSMA-targeting CAR is a first-generation CAR that can be stimulated in vitro by intracellular CD3z signaling domain alone.

In vivo, first generation CAR T cells are insufficient to induce an efficient T cell response and persistence and activity is limited (Brocker, T. & Karjalainen, K. J. Exp. Med. 181, 1653-1659 (1995)). In vivo, we expect to target primarily PSMA⁺/Nectin2⁺, and to a lesser extent PSMA⁺/Nectin2⁻, and spare PSMA⁺/Nectin2^{+/-} PCa cells. Further, as an indicator for successful T cell activation and function, IFNg secretion was assessed in the supernatant post co-culture using sandwich ELISA. As shown in FIG. 5F, the IFNg secretion levels of monospecific anti-PSMA, monospecific anti-Nectin2 and CombiCAR T cells correspond with their lysing capacities in presence of their target antigens, as assessed in FIG. 5E.

1-15. (canceled)

16. A nucleic acid that encodes:
a. a first chimeric antigen receptor (CAR) that comprises (i) an antigen binding domain that binds prostate-specific membrane antigen (PSMA), (ii) a transmembrane domain, and (iii) an intracellular signaling domain comprising an activation domain, and a second CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain, and a second CAR that comprises (i) an antigen binding domain that binds PSMA, (ii) a transmembrane domain, and (iii) an intracellular signaling domain comprising a co-stimulatory domain.

17. A pair of nucleic acids, comprising:
a. a first nucleic acid that encodes a CAR that comprises (i) an antigen binding domain that binds prostate-specific membrane antigen (PSMA), (ii) a transmembrane domain, and (iii) an intracellular signaling domain comprising an activation domain, and a second nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising an activation domain, and a second nucleic acid that encodes a CAR that comprises (i) an antigen binding domain that binds PSMA, (ii) a transmembrane domain, and (iii) an intracellular signaling domain comprising a co-stimulatory domain.

18. A pair of nucleic acids, comprising:
a. a first nucleic acid that encodes a synthetic Notch (synNotch) receptor comprising an antigen binding domain that binds prostate-specific membrane antigen (PSMA); and a second nucleic acid that encodes a chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain and optionally a co-stimulatory domain; or
b. a first nucleic acid that encodes a synthetic Notch (synNotch) receptor comprising an antigen binding domain that binds an antigen, other than PSMA, which is a marker of senescence in prostate cancer cells; and a second nucleic acid that encodes a chimeric antigen receptor (CAR) that comprises (i) an antigen binding domain that binds PSMA, (ii) a transmembrane domain, and (iii) an intracellular signaling domain comprising an activation domain and optionally a co-stimulatory domain;
wherein expression of the second nucleic acid that encodes a CAR is inducible in a cell upon binding of the synNotch receptor encoded by said first nucleic acid to (a.) PSMA or to (b.) the other antigen which is a marker of senescence in prostate cancer cells.

19. The nucleic acid according to claim 16, wherein the antigen binding domain is selected from the group consisting of an antibody, a Fv, a scFv, a Fab, or another antibody fragment.

20. The nucleic acid according to claim 16, wherein said transmembrane domain is a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.

21. The nucleic acid according to claim 16, wherein said activation domain and/or costimulatory domain is an intracellular domain from one or more protein selected from the group consisting of OX40, CD2, CD27, CD28, CD3 zeta, ICAM-1, LFA-1 (CD1 la/CD18), ICOS (CD278), and 4-1 BB (CD137).

22. The pair of nucleic acids according to claim 18, wherein the synNotch receptor comprises said antigen binding domain that binds PSMA or an antigen, other than PSMA, which is a marker of senescence in prostate cancer cells, fused to a Notch regulatory region, and a cleavable intracellular domain comprising a transcription factor, and the second nucleic acid that encodes a chimeric antigen receptor (CAR) under the control of a promoter that requires activation by the transcription factor comprised in the synNotch receptor.

23. A vector comprising a nucleic acid according to claim 16 or a pair of nucleic acids, wherein the pair of nucleic acids comprises:
a. a first nucleic acid that encodes a CAR that comprises (i) an antigen binding domain that binds prostate-specific membrane antigen (PSMA), (ii) a transmembrane domain, and (iii) an intracellular signaling domain comprising an activation domain, and a second nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain;
b. a first nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising an activation domain, and a second nucleic acid that encodes a CAR that comprises (i) an antigen binding domain that binds PSMA, (ii) a transmembrane domain, and (iii) an intracellular signaling domain comprising a co-stimulatory domain; or
c. a first nucleic acid that encodes a synthetic Notch (synNotch) receptor comprising an antigen binding domain that binds prostate-specific membrane antigen (PSMA); and a second nucleic acid that encodes a chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain and optionally a co-stimulatory domain; or
d. a first nucleic acid that encodes a synthetic Notch (synNotch) receptor comprising an antigen binding domain that binds an antigen, other than PSMA, which is a marker of senescence in prostate cancer cells; and a second nucleic acid that encodes a chimeric antigen receptor (CAR) that comprises (i) an antigen binding domain that binds PSMA, (ii) a transmembrane domain, and (iii) an intracellular signaling domain comprising an activation domain and optionally a co-stimulatory domain;
wherein expression of the second nucleic acid that encodes a CAR is inducible in a cell upon binding of the synNotch receptor encoded by said first nucleic acid to (c.) PSMA or to (d.) the other antigen which is a marker of senescence in prostate cancer cells.

24. An immunoresponsive cell that comprises a nucleic acid, a pair of nucleic acids, or a vector according to claim 23,
wherein the nucleic acid encodes:
a. a first chimeric antigen receptor (CAR) that comprises (i) an antigen binding domain that binds prostate-specific membrane antigen (PSMA), (ii) a transmembrane domain, and (iii) an intracellular signaling domain comprising an activation domain, and a second CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain, and a second CAR that comprises (i) an antigen binding domain that binds PSMA, (ii) a transmembrane domain, and (iii) an intracellular signaling domain comprising a co-stimulatory domain,
wherein the pair of nucleic acids comprises:
a. a first nucleic acid that encodes a CAR that comprises (i) an antigen binding domain that binds prostate-specific membrane antigen (PSMA), (ii) a transmembrane domain, and (iii) an intracellular signaling domain comprising an activation domain, and a second nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain;
b. a first nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising an activation domain, and a second nucleic acid that encodes a CAR that comprises (i) an antigen binding domain that binds PSMA, (ii) a transmembrane domain, and (iii) an intracellular signaling domain comprising a co-stimulatory domain;
c. a first nucleic acid that encodes a synthetic Notch (synNotch) receptor comprising an antigen binding domain that binds prostate-specific membrane antigen (PSMA); and a second nucleic acid that encodes a chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain and optionally a co-stimulatory domain; or
d. a first nucleic acid that encodes a synthetic Notch (synNotch) receptor comprising an antigen binding domain that binds an antigen, other than PSMA, which is a marker of senescence in prostate cancer cells; and a second nucleic acid that encodes a chimeric antigen receptor (CAR) that comprises (i) an antigen binding domain that binds PSMA, (ii) a transmembrane domain, and (iii) an intracellular signaling domain comprising an activation domain and optionally a co-stimulatory domain;
wherein expression of the second nucleic acid that encodes a CAR is inducible in a cell upon binding of the synNotch receptor encoded by said first nucleic acid to (c.) PSMA or to (d.) the other antigen which is a marker of senescence in prostate cancer cells.

25. The immunoresponsive cell according to claim 24, which comprises:
a. a first CAR that comprises (i) an antigen binding domain that binds prostate-specific membrane antigen (PSMA), (ii) a transmembrane domain, and (iii) an intracellular signaling domain comprising an activation domain, and a second CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first CAR that comprises (i) an antigen binding domain that binds an antigen, other than PSMA, which is a marker of senescence in prostate cancer cells, (ii) a transmembrane domain, and (iii) an intracellular signaling domain comprising an activation domain, and a second CAR that comprises (i) an antigen binding domain that binds PSMA, (ii) a transmembrane domain, and (iii) an intracellular signaling domain comprising a co-stimulatory domain.

26. The immunoresponsive cell according to claim 24, which comprises:
a. a synthetic Notch (synNotch) receptor comprising an antigen binding domain that binds prostate-specific membrane antigen (PSMA); and a nucleic acid that encodes a chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain and optionally a co-stimulatory domain; or
b. a synNotch receptor comprising an antigen binding domain that binds an antigen, other than PSMA, which is a marker of senescence in prostate cancer cells; and a nucleic acid that encodes a chimeric antigen receptor (CAR) that comprises (i) an antigen binding domain that binds PSMA, (ii) a transmembrane domain, and (iii) an intracellular signaling domain comprising an activation domain and optionally a co-stimulatory domain;
wherein expression of the nucleic acid that encodes a CAR is inducible in a cell upon binding of the synNotch receptor encoded by said first nucleic acid to (a.) PSMA or to (b.) the other antigen which is a marker of senescence in prostate cancer cells.

27. The immunoresponsive cell according to claim 24, wherein said immunoresponsive cell is activated only upon binding to both PSMA and said antigen, other than PSMA, which is a marker of senescence in prostate cancer cells.

28. The nucleic acid according to claim 16, wherein the antigen, other than PSMA, which is a marker of senescence in prostate cancer cells is Nectin2.

29. A method of treating a subject in need thereof, comprising administering to the subject a therapeutically effective amount of a nucleic acid, a pair of nucleic acids, a vector comprising the nucleic acid, a vector comprising the pair of nucleic acids, or an immunoresponsive cell according to claim 24,
wherein the nucleic acid encodes:
a. a first chimeric antigen receptor (CAR) that comprises (i) an antigen binding domain that binds prostate-specific membrane antigen (PSMA), (ii) a transmembrane domain, and (iii) an intracellular signaling domain comprising an activation domain, and a second CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain, and a second CAR that comprises (i) an antigen binding domain that binds PSMA, (ii) a transmembrane domain, and (iii) an intracellular signaling domain comprising a co-stimulatory domain,
wherein the pair of nucleic acids comprises:
a. a first nucleic acid that encodes a CAR that comprises (i) an antigen binding domain that binds prostate-specific membrane antigen (PSMA), (ii) a transmembrane domain, and (iii) an intracellular signaling domain comprising an activation domain, and a second nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain;
b. a first nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising an activation domain, and a second nucleic acid that encodes a CAR that comprises (i) an antigen binding domain that binds PSMA, (ii) a transmembrane domain, and (iii) an intracellular signaling domain comprising a co-stimulatory domain;
c. a first nucleic acid that encodes a synthetic Notch (synNotch) receptor comprising an antigen binding domain that binds prostate-specific membrane antigen (PSMA); and a second nucleic acid that encodes a chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain and optionally a co-stimulatory domain; or
d. a first nucleic acid that encodes a synthetic Notch (synNotch) receptor comprising an antigen binding domain that binds an antigen, other than PSMA, which is a marker of senescence in prostate cancer cells; and a second nucleic acid that encodes a chimeric antigen receptor (CAR) that comprises (i) an antigen binding domain that binds PSMA, (ii) a transmembrane domain, and (iii) an intracellular signaling domain comprising an activation domain and optionally a co-stimulatory domain;
wherein expression of the second nucleic acid that encodes a CAR is inducible in a cell upon binding of the synNotch receptor encoded by said first nucleic acid to (c.) PSMA or to (d.) the other antigen which is a marker of senescence in prostate cancer cells.

30. A method of treating prostate cancer in a subject in need thereof, comprising administering to the subject a therapeutically effective amount of a nucleic acid, a pair of nucleic acids, a vector comprising the nucleic acid, a vector comprising the pair of nucleic acids, or an immunoresponsive cell according to claim 24,
wherein the nucleic acid encodes:
a. a first chimeric antigen receptor (CAR) that comprises (i) an antigen binding domain that binds prostate-specific membrane antigen (PSMA), (ii) a transmembrane domain, and (iii) an intracellular signaling domain comprising an activation domain, and a second CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain; or
b. a first chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain, and a second CAR that comprises (i) an antigen binding domain that binds PSMA, (ii) a transmembrane domain, and (iii) an intracellular signaling domain comprising a co-stimulatory domain,
wherein the pair of nucleic acids comprises:
a. a first nucleic acid that encodes a CAR that comprises (i) an antigen binding domain that binds prostate-specific membrane antigen (PSMA), (ii) a transmembrane domain, and (iii) an intracellular signaling domain comprising an activation domain, and a second nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising a co-stimulatory domain;
b. a first nucleic acid that encodes a CAR that comprises (i) an antigen binding domain that binds an antigen, other than PSMA, which is a marker of senescence in prostate cancer cells, (ii) a transmembrane domain, and (iii) an intracellular signaling domain comprising an activation domain, and a second nucleic acid that encodes a CAR that comprises (i) an antigen binding domain that binds PSMA, (ii) a transmembrane domain, and (iii) an intracellular signaling domain comprising a co-stimulatory domain;
c. a first nucleic acid that encodes a synthetic Notch (synNotch) receptor comprising an antigen binding domain that binds prostate-specific membrane antigen (PSMA); and a second nucleic acid that encodes a chimeric antigen receptor (CAR) that comprises (i) an antigen binding domain that binds an antigen, other than PSMA, which is a marker of senescence in prostate cancer cells, (ii) a transmembrane domain, and (iii) an intracellular signaling domain comprising an activation domain and optionally a co-stimulatory domain; or
d. a first nucleic acid that encodes a synthetic Notch (synNotch) receptor comprising an antigen binding domain that binds an antigen, other than PSMA, which is a marker of senescence in prostate cancer cells; and a second nucleic acid that encodes a chimeric antigen receptor (CAR) that comprises (i) an antigen binding domain that binds PSMA, (ii) a transmembrane domain, and (iii) an intracellular signaling domain comprising an activation domain and optionally a co-stimulatory domain;
wherein expression of the second nucleic acid that encodes a CAR is inducible in a cell upon binding of the synNotch receptor encoded by said first nucleic acid to (c.) PSMA or to (d.) the other antigen which is a marker of senescence in prostate cancer cells.
